SEQUENCE LISTING (5 sequences)

SEQ ID NO: 1 - protein, 894 aa, Homo sapiens
MSRRKQGNPQHLSQRELITPEADHVEAAILEEDEGLEIEEPSGLGLMVGGPDPDLLTCGQCQMNFPLGDILVFIEHKRKQCGGSLGACYDKALDKDSPPPSSRSELRKVSEPVEIGIQVTPDEDDHLLSPTKGICPKQENIAGPCRPAQLPAVAPIAASSHPHSSVITSPLRALGALPPCLPLPCCSARPVSGDGTQGEGQTEAPFGCQCQLSGKDEPSSYICTTCKQPFNSAWFLLQHAQNTHGFRIYLEPGPASSSLTPRLTIPPPLGPEAVAQSPLMNFLGDSNPFNLLRMTGPILRDHPGFGEGRLPGTPPLFSPPPRHHLDPHRLSAEEMGLVAQHPSAFDRVMRLNPMAIDSPAMDFSRRLRELAGNSSTPPPVSPGRGNPMHRLLNPFQPSPKSPFLSTPPLPPMPPGGTPPPQPPAKSKSCEFCGKTFKFQSNLIVHRRSHTGEKPYKCQLCDHACSQASKLKRHMKTHMHKAGSLAGRSDDGLSAASSPEPGTSELAGEGLKAADGDFRHHESDPSLGHEPEEEDEEEEEEEEELLLENESRPESSFSMDSELSRNRENGGGGVPGVPGAGGGAAKALADEKALVLGKVMENVGLGALPQYGELLADKQKRGAFLKRAAGGGDAGDDDDAGGCGDAGAGGAVNGRGGGFAPGTEPFPGLFPRKPAPLPSPGLNSAAKRIKVEKDLELPPAALIPSENVYSQWLVGYAASRHFMKDPFLGFTDARQSPFATSSEHSSENGSLRFSTPPGDLLDGGLSGRSGTASGGSTPHLGGPGPGRPSSKEGRRSDTCEYCGKVFKNCSNLTVHRRSHTGERPYKCELCNYACAQSSKLTRHMKTHGQIGKEVYRCDICQMPFSVYSTLEKHMKKWHGEHLLTNDVKIEQAERS

SEQ ID NO: 2 - DNA, 2685 bp, Homo sapiens
atgtcccgccgcaaacagggcaacccgcagcacttgtcccagagggagctcatcaccccagaggctgaccatgtggaggccgccatcctcgaagaagacgagggtctggagatagaggagccaagtggcctggggctgatggtgggtggccccgaccctgacctgctcacctgtggccagtgtcaaatgaacttccccttgggggacatcctggtttttatagagcacaaaaggaagcagtgtggcggcagcttgggtgcctgctatgacaaggccctggacaaggacagcccgccaccctcctcacgctccgagctcaggaaagtgtccgagccggtggagatcgggatccaagtcacccccgacgaagatgaccacctgctctcacccacgaaaggcatctgtcccaagcaggagaacattgcagggccgtgcaggcctgcccagctgccagcggtggcccccatagctgcctcctcccaccctcactcatccgtgatcacttcacctctgcgtgccctgggcgctctcccgccctgcctccccctgccgtgctgcagcgcgcgcccggtctcgggtgacgggactcagggtgagggtcagacggaggctccctttggatgccagtgtcagttgtcaggtaaagatgagccttccagctacatttgcacaacatgcaagcagcccttcaacagcgcgtggttcctgctgcagcacgcgcagaacacgcacggcttccgcatctacctggagcccgggccggccagcagctcgctcacgccgcggctcaccatcccgccgccgctcgggccggaggccgtggcgcagtccccgctcatgaatttcctgggcgacagcaaccccttcaacctgctgcgcatgacgggccccatcctgcgggaccacccgggcttcggcgagggccgcctgccgggcacgccgcctctcttcagtcccccgccgcgccaccacctggacccgcaccgcctcagtgccgaggagatggggctcgtcgcccagcaccccagtgccttcgaccgagtcatgcgcctgaaccccatggccatcgactcgcccgccatggacttctcgcggcggctccgcgagctggcgggcaacagctccacgccgccgcccgtgtccccgggccgcggcaaccctatgcaccggctcctgaaccccttccagcccagccccaagtccccgttcctgagcacgccgccgctgccgcccatgccccctggcggcacgccgcccccgcagccgccagccaagagcaagtcgtgcgagttctgcggcaagaccttcaagttccagagcaatctcatcgtgcaccggcgcagtcacacgggcgagaagccctacaagtgccagctgtgcgaccacgcgtgctcgcaggccagcaagctcaagcgccacatgaagacgcacatgcacaaggccggctcgctggccggccgctccgacgacgggctctcggccgccagctcccccgagcccggcaccagcgagctggcgggcgagggcctcaaggcggccgacggtgacttccgccaccacgagagcgacccgtcgctgggccacgagccggaggaggaggacgaggaggaggaggaggaggaggaggagctgctactggagaacgagagccggcccgagtcgagcttcagcatggactcggagctgagccgcaaccgcgagaacggcggtggtggggtgcccggggtcccgggcgcggggggcggcgcggccaaggcgctggctgacgagaaggcgctggtgctgggcaaggtcatggagaacgtgggcctaggcgcactgccgcagtacggcgagctcctggccgacaagcagaagcgcggcgccttcctgaagcgtgcggcgggcggcggggacgcgggcgacgacgacgacgcgggcggctgcggggacgcgggcgcgggcggcgcggtcaacgggcgcgggggcggcttcgcgccaggcaccgagcccttccccgggctcttcccgcgcaagcccgcgccgctgcccagccccgggctcaacagcgccgccaagcgcatcaaggtggagaaggacctggagctgccgcccgccgcgctcatcccgtccgagaacgtgtactcgcagtggctggtgggctacgcggcgtcgcggcacttcatgaaggaccccttcctgggcttcacggacgcacgacagtcgcccttcgccacgtcgtccgagcactcgtccgagaacggcagcctgcgcttctccacgccgcccggggacctgctggacggcggcctctcgggccgcagcggcacggccagcggaggcagcaccccgcacctgggcggcccgggccccgggcggcccagctccaaggagggccgccgcagcgacacgtgcgagtactgcggcaaggtgttcaagaactgcagcaacttgacggtgcaccggcggagccacaccggcgagcggccttacaagtgcgagctgtgcaactacgcgtgcgcgcagagcagcaagctcacgcgccacatgaagacgcacgggcagatcggcaaggaggtgtaccgctgcgacatctgccagatgcccttcagcgtctacagcaccctggagaaacacatgaaaaagtggcacggcgagcacttgctgactaacgacgtcaaaatcgagcaggccgagaggagctaa

SEQ ID NO: 3 - DNA, 536 bp, Artificial sequence
  Synthetic nucleic acid
atcgattagtccaatttgttaaagacaggatatcagtggtccaggctctagttttgactcaacaatatcaccagctgaagcctatagagtacgagccatagatagaataaaagattttatttagtctccagaaaaaggggggaatgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgatc

SEQ ID NO: 4 - DNA, 2472 bp, Homo sapiens
atgtcccgccgcaaacagggcaacccgcagcacttgtcccagagggagctcatcaccccagaggctgaccatgtggaggccgccatcctcgaagaagacgagggtctggagatagaggagccaagtggcctggggctgatggtgggtggccccgaccctgacctgctcacctgtggccagtgtcaaatgaacttccccttgggggacatcctggtttttatagagcacaaaaggaagcagtgtggcggcagcttgggtgcctgctatgacaaggccctggacaaggacagcccgccaccctcctcacgctccgagctcaggaaagtgtccgagccggtggagatcgggatccaagtcacccccgacgaagatgaccacctgctctcacccacgaaaggcatctgtcccaagcaggagaacattgcaggtaaagatgagccttccagctacatttgcacaacatgcaagcagcccttcaacagcgcgtggttcctgctgcagcacgcgcagaacacgcacggcttccgcatctacctggagcccgggccggccagcagctcgctcacgccgcggctcaccatcccgccgccgctcgggccggaggccgtggcgcagtccccgctcatgaatttcctgggcgacagcaaccccttcaacctgctgcgcatgacgggccccatcctgcgggaccacccgggcttcggcgagggccgcctgccgggcacgccgcctctcttcagtcccccgccgcgccaccacctggacccgcaccgcctcagtgccgaggagatggggctcgtcgcccagcaccccagtgccttcgaccgagtcatgcgcctgaaccccatggccatcgactcgcccgccatggacttctcgcggcggctccgcgagctggcgggcaacagctccacgccgccgcccgtgtccccgggccgcggcaaccctatgcaccggctcctgaaccccttccagcccagccccaagtccccgttcctgagcacgccgccgctgccgcccatgccccctggcggcacgccgcccccgcagccgccagccaagagcaagtcgtgcgagttctgcggcaagaccttcaagttccagagcaatctcatcgtgcaccggcgcagtcacacgggcgagaagccctacaagtgccagctgtgcgaccacgcgtgctcgcaggccagcaagctcaagcgccacatgaagacgcacatgcacaaggccggctcgctggccggccgctccgacgacgggctctcggccgccagctcccccgagcccggcaccagcgagctggcgggcgagggcctcaaggcggccgacggtgacttccgccaccacgagagcgacccgtcgctgggccacgagccggaggaggaggacgaggaggaggaggaggaggaggaggagctgctactggagaacgagagccggcccgagtcgagcttcagcatggactcggagctgagccgcaaccgcgagaacggcggtggtggggtgcccggggtcccgggcgcggggggcggcgcggccaaggcgctggctgacgagaaggcgctggtgctgggcaaggtcatggagaacgtgggcctaggcgcactgccgcagtacggcgagctcctggccgacaagcagaagcgcggcgccttcctgaagcgtgcggcgggcggcggggacgcgggcgacgacgacgacgcgggcggctgcggggacgcgggcgcgggcggcgcggtcaacgggcgcgggggcggcttcgcgccaggcaccgagcccttccccgggctcttcccgcgcaagcccgcgccgctgcccagccccgggctcaacagcgccgccaagcgcatcaaggtggagaaggacctggagctgccgcccgccgcgctcatcccgtccgagaacgtgtactcgcagtggctggtgggctacgcggcgtcgcggcacttcatgaaggaccccttcctgggcttcacggacgcacgacagtcgcccttcgccacgtcgtccgagcactcgtccgagaacggcagcctgcgcttctccacgccgcccggggacctgctggacggcggcctctcgggccgcagcggcacggccagcggaggcagcaccccgcacctgggcggcccgggccccgggcggcccagctccaaggagggccgccgcagcgacacgtgcgagtactgcggcaaggtgttcaagaactgcagcaacttgacggtgcaccggcggagccacaccggcgagcggccttacaagtgcgagctgtgcaactacgcgtgcgcgcagagcagcaagctcacgcgccacatgaagacgcacgggcagatcggcaaggaggtgtaccgctgcgacatctgccagatgcccttcagcgtctacagcaccctggagaaacacatgaaaaagtggcacggcgagcacttgctgactaacgacgtcaaaatcgagcaggccgagaggagctaa

SEQ ID NO: 5 - protein, 823 aa, Homo sapiens
MSRRKQGNPQHLSQRELITPEADHVEAAILEEDEGLEIEEPSGLGLMVGGPDPDLLTCGQCQMNFPLGDILVFIEHKRKQCGGSLGACYDKALDKDSPPPSSRSELRKVSEPVEIGIQVTPDEDDHLLSPTKGICPKQENIAGKDEPSSYICTTCKQPFNSAWFLLQHAQNTHGFRIYLEPGPASSSLTPRLTIPPPLGPEAVAQSPLMNFLGDSNPFNLLRMTGPILRDHPGFGEGRLPGTPPLFSPPPRHHLDPHRLSAEEMGLVAQHPSAFDRVMRLNPMAIDSPAMDFSRRLRELAGNSSTPPPVSPGRGNPMHRLLNPFQPSPKSPFLSTPPLPPMPPGGTPPPQPPAKSKSCEFCGKTFKFQSNLIVHRRSHTGEKPYKCQLCDHACSQASKLKRHMKTHMHKAGSLAGRSDDGLSAASSPEPGTSELAGEGLKAADGDFRHHESDPSLGHEPEEEDEEEEEEEEELLLENESRPESSFSMDSELSRNRENGGGGVPGVPGAGGGAAKALADEKALVLGKVMENVGLGALPQYGELLADKQKRGAFLKRAAGGGDAGDDDDAGGCGDAGAGGAVNGRGGGFAPGTEPFPGLFPRKPAPLPSPGLNSAAKRIKVEKDLELPPAALIPSENVYSQWLVGYAASRHFMKDPFLGFTDARQSPFATSSEHSSENGSLRFSTPPGDLLDGGLSGRSGTASGGSTPHLGGPGPGRPSSKEGRRSDTCEYCGKVFKNCSNLTVHRRSHTGERPYKCELCNYACAQSSKLTRHMKTHGQIGKEVYRCDICQMPFSVYSTLEKHMKKWHGEHLLTNDVKIEQAERS

(19) United States
(12) Patent Application Publication
(10) Pub. No.: US 2022/0347222 A1
(43) Pub. Date: Nov. 3, 2022

(54) BCL11B Overexpression to Enhance Human Thymopoiesis and T Cell Function

(71) Applicants: The Regents of the University of California, Oakland, CA (US); Children's Hospital Los Angeles, Los Angeles, CA (US)

(72) Inventors: Chintan PAREKH, Los Angeles, CA (US); Gay CROOKS, Los Angeles, CA (US); Christopher SEETS, Los Angeles, CA (US); Amelie Montel-Hagen, Los Angeles, CA (US)

(73) Assignees: The Regents of the University of California, Oakland, CA (US); Children's Hospital Los Angeles, Los Angeles, CA (US)

(21) Appl. No.: 17/620,603

(22) PCT Filed: Jun. 24, 2020
(86) PCT No.: PCT/US2020/039414

§ 371 Date: Dec. 17, 2021

Related U.S. Application Data
(60) Provisional application No. 62/865,835, filed on Jun. 24, 2019.

(51) Int. Cl. A61K 35/28; A61K 35/545; A61K 35/17; A61K 38/17

(52) U.S. Cl. CPC A61K 35/28; A61K 35/545; A61K 35/17; A61K 38/1774; A61K 38/177

(57) ABSTRACT

Methods of treating a subject using a T cell therapy are disclosed herein. The methods include increasing BCL11B expression in hematopoietic stem and progenitor cells (HSPCs), pluripotent stem cells, or mature T cells to form modified cells and administering a therapeutically effective amount of the modified cells to the subject for the T cell therapy. BCL11B expression in the HSPCs, pluripotent stem cells, or mature T cells increases production and/or proliferation of T cells from the HSPCs and/or the pluripotent stem cells, and/or increases proliferation of the T cells.

### CROSS REFERENCE TO RELATED APPLICATION

This application claims the benefit of U.S. Provisional Application No. 62/865,835 filed Jun. 24, 2019, which is incorporated by reference herein in its entirety.

### ACKNOWLEDGMENT OF GOVERNMENT SUPPORT

This invention was made with Government support under K12-HD052954 awarded by the National Institutes of Health. The Government has certain rights in the invention.

### FIELD

This relates to methods of producing a T cell population for a T cell therapy, as well as to treating a subject using a T cell therapy.

### BACKGROUND

Over eight thousand children and adults undergo allogenic hematopoietic stem cell transplant (HSCT) in the United States each year, a curative therapy for several benign and malignant hematological diseases and genetic disorders. However, a major continued challenge has been the significant morbidity and mortality from serious infections and recurrence of malignant disease related to the slow and deficient recovery of T cell immunity following HSCT. Recovery of T cell immunity following allogenic HSCT takes 1-2 years, and many patients show even longer-term T cell function deficits. T cell reconstitution occurs in the initial months after HSCT through the expansion of transplanted mature donor T cells to generate a pool with limited T cell receptor (TCR) diversity. However, the recovery of long-term T cell immunity with a broad TCR repertoire takes many months and occurs through the generation of new T cells from donor hematopoietic stem and progenitor cells (HSPC) that migrate to the thymus (thymopoiesis).

Further, engineered T-cell immunotherapies have shown promising remission rates in acute leukemias and lymphomas but exhaustion or lack of persistence of the infused T-cells results in disease relapse in many cases. Furthermore, the poor function of the infused T-cells in the tumor microenvironment has severely limited the efficacy of engineered T-cells for solid malignancies.

### SUMMARY

Methods of producing a T cell population for a T cell therapy and of treating a subject using a T cell therapy are disclosed herein.

In some embodiments a method of treating a subject with a T cell therapy is provided. The method includes providing HSPCs, pluripotent stem cells, or mature T cells, and increasing BCL11B expression in the HSPCs, pluripotent stem cells, or mature T cells to produce modified cells with increased BCL11B expression compared to corresponding control cells. The increased BCL11B expression increases production and/or proliferation of T cells from the HSPCs or the pluripotent stem cells, or increases production and/or proliferation of the mature T cells, compared to the corresponding control cells. A therapeutically effective amount of the modified cells is administered to the subject for the T cell therapy. In some such embodiments, the subject is a HSCT patient and the T cell therapy comprises thymic T cell reconstitution in the subject following the HSCT. In additional embodiments, the subject is a cancer patient and the T cell therapy is a chimeric antigen receptor (CAR) T cell therapy or an engineered T cell receptor (TCR) T cell therapy for treatment of the cancer. In some such embodiments, the method further comprises transducing the HSPCs, pluripotent stem cells, mature T cells, or the modified cells with a heterologous nucleic acid molecule encoding the CAR or the TCR before administering the modified cells to a subject.

In additional embodiments, a method of producing a T cell population for a T cell therapy for a human subject is provided. The method includes providing HSPCs, pluripotent stem cells, or mature T cells, and increasing BCL11B expression in the HSPCs, pluripotent stem cells, or mature T cells to form modified cells with increased BCL11B expression compared to corresponding control cells. The increased BCL11B expression increases production and/or proliferation of T cells from the HSPCs or the pluripotent stem cells, or increases proliferation of the mature T cells, compared to the corresponding control cells, to form the T cell population for the T cell therapy. In several such embodiments, the modified cells are incubated in vitro (such as for more than 14 days or more than 30 days) under conditions sufficient for differentiation, production, and/or proliferation of T cells from the HSPCs and/or pluripotent stem cells, or proliferation of the mature T cells. In some embodiments, the T cell population is a population of HSPC for administration to a HSCT patient for thymic T cell reconstitution in the subject following the HSCT. In additional embodiments, the T cell population comprises CAR T cells or TCR T cells and the T cell therapy is a CAR T cell therapy or a TCR T cell therapy. In several such embodiments, the method further comprises transducing the HSPCs, pluripotent stem cells, mature T cells, or the modified cells with a heterologous nucleic acid molecule encoding the CAR or the TCR before administering the cells to a subject.

In some embodiments, BCL11B expression in the HSPCs, pluripotent stem cells, or mature T cells is increased by transducing the cells with a heterologous nucleic acid encoding BCL11B. In certain embodiments, the cells are transduced with a viral vector, such as a lentiviral vector, comprising the nucleic acid encoding for BCL11B, which is operably linked to a promoter, such as an MND or MSCV promoter. In some embodiments, the BCL11B expression in the modified cells is at least that of a positive control cell, such as a CD34+ or CD34−CD4+CD8+ human thymic T-cell precursor. In certain embodiments, the modified cells are mature T cells with BCL11B expression 2 to 10-fold higher than BCL11B expression in the corresponding control mature T cells without the increase in BCLB11B expression.

In some embodiments, T cells proliferating from the modified cells have delayed exhaustion, an increased central memory immunophenotype, and/or increased interleukin 2 production and/or TNF-alpha production compared to corresponding control cells. In certain disclosed embodiments, the T cells with an enhanced central memory immunophenotype may be CD45RO+CD62L+CCR7+ T cells, and T cell production and/or proliferation from the modified cells may be independent of Notch signaling.

The foregoing and other features and advantages of this disclosure will become more apparent from the following detailed description of several embodiments which proceeds with reference to the accompanying figures.

### BRIEF DESCRIPTION OF THE FIGURES

FIG. 1 shows a schematic illustrating stages of human thymopoiesis. BM: bone marrow; HPC: hematopoietic progenitor cells, ISP: immature single positive cells.

FIG. 2 shows a graph illustrating expression profiles of BCL11B and TCF7 during human thymopoiesis. BCL11B and TCF7 mRNA expression (RNA-Seq data) in human bone marrow hematopoietic stem cells and thymic populations (mean, SEM, n=2 biological replicates per cell type, Thy1 vs Thy4 FDR adjusted p values<0.05 for both genes). FPKM: Fragments per kilobase per million reads. HSC: hematopoietic stem cells (CD34+CD38−); Thy1: CD34+CD7−CD1a−; Thy2: CD34+CD7−CD1a−; Thy3: CD34+CD7−CD1a+; Thy4: CD4+CD8+ cells.

FIGS. 3A-3F show FACS analysis results and graphs illustrating that BCL11B gain of function enhances T-lineage differentiation of human HSPCs. CD34+ cord blood (CB) HSPC transduced with BCL11B-GFP (BCL11B) or control GFP (Ctrl) lentivirus were cultured in artificial thymic organoids (ATO) (2,000-5000 FACS sorted CD34+GFP+ cells/ATO, in vitro T-cell differentiation system). (FIG. 3A) FACS gates for sorting CD34+GFP+ cells. (FIG. 3B) FACS of cultures at serial timepoints (pre-gated on CD45+GFP+ cells, representative data from 9 experiments, each experiment done with a different CB pool). (FIG. 3C) Kinetics of T-cell differentiation (data from 9 experiments, each with a different CB pool), % CD3-CD4+CD8+ cells shown as an example, BCL11B HSPC have significantly accelerated differentiation (p<0.05, BCL11B vs Ctrl) Second order polynomial regressions of the logit of proportions of different stages vs time (curves) and individual data-points for proportions of different stages shown. (FIG. 3D) Cell counts of committed T-precursors (CD7+CD1a+), CD4+CD8+ and CD8+SP cells, p<0.05 for BCL11B (FIG. 3B) vs Ctrl (FIG. 3C), mean, SEM (n=5-6 experiments, each with a different CB pool). (FIG. 3E) FACS of CD8 single positive (SP) cells arising from BCL11B HSPC showing naïve mature T-cell phenotype (3+TCRαβ+45RA+CCR7+62L+1a−). (FIG. 3F) Week 12 flow cytometry analysis of ATOs (pre-gated on CD45+GFP+ cells, representative data from one of two experiments, each experiment done with a different CB pool).

FIGS. 4A-4C show FACS analysis results and graphs illustrating that T-cells derived from BCL11B overexpressing HSPC exhibit enhanced proliferation and differentiation into cells with a central memory immunophenotype. Naïve T-cells sorted at 6-12 weeks from ATO cultures in FIG. 2 were stimulated with anti-CD3/CD28 beads and re-cultured in the presence of IL-2 (stimulated on day 0 and 10). (FIG. 4A) FACS strategy for sorting naïve mature T-cells from ATO cultures prior to stimulation. (FIG. 4B) Flow cytometry analysis of cultures in (FIG. 4B) on day 10 post-ATO stimulation for assessment of frequency of cells with a central memory immunophenotype (CCR7+CD62L+CD45RO+). Mean and SEM shown. P<0.05 for BCL11B vs control. (FIG. 4C) Cell counts in culture following stimulation with anti-CD3/CD28 beads. P<0.001 for BCL11B vs control. N=4 experiments for (FIG. 4B) and (FIG. 4C). Experiments 1 and 2 and Experiments 3 and 4 were done with cells from separate CB donor pools respectively (i.e. n=2 CB donor pools).

FIGS. 5A-5F show FACS analysis results and graphs illustrating that BCL11B overexpression enhances the function of peripheral blood T cells and prolongs the anti-cancer effect of CAR T cells in vitro. (FIGS. 5A-5D) T-cells isolated from human peripheral blood (PBTC) were transduced with BCL11B (isoform 1)-GFP (BCL11B1), BCL11B (isoform 2)-GFP (BCL11B2), or control GFP (Ctrl) lentivirus. GFP+ cells were sorted and used in FIGS. 5A-5D. (FIG. 5A) qPCR for expression of isoform 1 (B1), isoform 2 (B2) and total BCL11B (B) in BCL11B1 (B1), BCL11B2 (B2), control, and untransduced (UT) cells. (FIG. 5B) cytokine production (PMA stimulation). (FIGS. 5C-5D) FACS for central memory immunophenotype (CCR7+CD62L+, upper plots, all cells were CD45RO+) and exhaustion markers (lower plots) (FIG. 5C), and proliferation (FIG. 5D) following recurrent CD3/CD28 stimulation of sorted cells (stimulated on days 0,10,20) (In FIGS. 5A-5D, two independent experiments for BCL11B1 and one experiment for BCL11B2 were done, each in triplicate, one representative experiment shown. Each experiment was done with a different donor). T-cells transduced with BCL11B vector at MOI=10 failed to proliferate in response to CD3/CD28 stimulation. (FIGS. 5E-5F) 30,000 PBTC transduced with CD19 chimeric antigen receptor (CAR) lentivirus (CD19) or co-transduced with CD19 CAR and BCL11B1 lentiviruses (CD19-B) were co-cultured with 30,000 CD19+ acute lymphoblastic leukemia (ALL) cells (1:1 effector target ratio) and then restimulated with fresh ALL cells on days 5, 9, 14, and 20 (1 experiment in triplicate). ALL cell counts (FIG. 5E) and FACS for CD19+ ALL and CD3+ T-cells on day 5 and day 20 of culture (FIG. 5F). Error bars (FIGS. 5B, 5D, 5E): SEM.

FIGS. 6A-6B show a mathematical model and graphs illustrating that BCL11B overexpressing cells exhibit accelerated differentiation at multiple cell state transitions during T-cell differentiation. Proliferation, death, and cell state transition rates in ATO cultures initiated with cord blood (CB) HSPC transduced with BCL11B-GFP (BCL11B) or control GFP (Ctrl) lentivirus were mathematically modeled. (FIG. 6A) Differentiation stages, parameters (b, d, t, K) and differential equations included in the mathematical model. S1-6: stages of T-cell differentiation. S1: CD4−CD8− (double negative, DN, S2: CD4+CD8−CD3− (immature single positive, ISP); S3: CD4+CD8+CD3− (early double positive, CD3− DP), S4: CD4+CD8+CD3+(late double positive, CD3+DP); S5: CD4+CD8−CD3+(CD4 single positive, CD4SP); S6: CD4−CD8+CD3+(CD8 single positive, CD8SP). Proliferation and transition rates predicted by the model to be increased in BCL11B ATOs are shown with black arrows. K: maximal cell capacity of an ATO. (FIG. 6B) Modeled kinetics for cell counts of cells at different stages of differentiation in BCL11B and control ATOs.

FIGS. 7A-7D show a diagram and graphs illustrating that BCL11B overexpression in HSPC acutely induces a T-cell transcriptional program and represses alternative lineage programs. CD34+ cord blood (CB) HSPC transduced with BCL11B-GFP (BCL11B) or control GFP (control) lentivirus were sorted for RNA-Seq. (FIG. 7A) Diagram illustrating the experimental scheme. (FIGS. 7A-7B) Enrichment of genes upregulated in BCL11B or control cells among genes ranked by CD34+CD7−CD1a− (Thy1) vs. CD34+CD7+CD1a+(Thy3) (FIG. 7B) or BCL11B knockdown vs scramble control (FIG. 7C) expression. B_up, C_up: genes upregulated in BCL11B or control cells respectively in a multivariate BCL11B vs control differential expression analysis (FDR<0.05) that included CB donor and timepoint (48 hours or 7 days) as co-variates. B7_up, C7_up: genes upregulated in BCL11B or control cells sorted from ATOs on day 7 (FDR<0.05). NES: normalized enrichment score. FDR: False discovery rate adjusted p value. (FIG. 7D) Fold changes for a subset of genes known to be associated with stem/progenitor cells or lineage differentiation in hematopoiesis (FDR<0.05 for these genes in the multivariate analysis in FIG. 7C). Positive fold change: upregulated in BCL11B cells. Negative Fold change: upregulated in control cells.

FIG. 8A-8D show FACS analysis results and graphs illustrating that BCL11B is sufficient for the initiation of T-lineage differentiation and can inhibit myeloid differentiation in the absence of NOTCH signaling. Cord blood (CB) HSPC transduced with BCL11B-GFP (BCL11B) or control GFP (Ctrl) lentivirus were cultured in MS5 organoids (No NOTCH signaling) or MS5-DLL1 ATOs (NOTCH1 signaling). (FIG. 8A) FACS analysis (day 20 of culture); (FIG. 8B) % T cell precursors (CD5+CD7+CD56−); and (FIG. 8C) % myeloid (CD33+) cells in cultures (mean, SEM, paired t test, 3 experiments, each with a different CB pool. (FIG. 8D) Gene expression (qPCR) in CD5+CD7+ cells sorted from culture (day 12, CD45+ cells sorted for ctrl, MS5, 0=expression not detected), 1 of 2 experiments shown.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file in the form of the file named “Sequence.txt” (˜22 KB), which was created on Jun. 23, 2020, which is incorporated by reference herein.

SEQ ID NO: 1 is the amino acid sequence of BCL11B isoform 1.

SEQ ID NO: 2 is an exemplary nucleotide sequence encoding BCL11B isoform 1.

SEQ ID NO: 3 is an MND promoter.

SEQ ID NO: 4 is an exemplary nucleotide sequence encoding BCL11B isoform 2.

SEQ ID NO: 5 is the amino acid sequence of BCL11B isoform 2.

### DETAILED DESCRIPTION

### I. Introduction

Strategies to enhance T-cell differentiation and function are critically needed in at least three clinical areas: 1) to improve outcomes of patients treated with cellular therapies like bone marrow transplantation; 2) to improve the efficacy of engineered T-cell immunotherapies for cancer; and 3) enable the generation of T-cells from pluripotent stem cells for immunotherapy applications. While bone marrow transplantation is a curative treatment for benign and malignant hematological diseases, morbidity and mortality from delayed thymic T-cell reconstitution continues to be a significant clinical problem. Engineered T-cell immunotherapies have shown promising remission rates in acute leukemias and lymphomas but exhaustion or lack of persistence of the infused T-cells results in disease relapse in many cases. Furthermore, the poor function of the infused T-cells in the tumor microenvironment has severely limited the efficacy of engineered T-cells for solid malignancies.

HSPCs and pluripotent stem cells represent an attractive source of off-the-shelf allogenic T-cells for immunotherapy. However, the lack of efficient technologies to generate adequate numbers of T cells from these precursor cells remains a significant obstacle to the clinical translation of HSPCs and pluripotent stem cell derived T-cell immunotherapies.

To meet these needs, and as disclosed herein for the first time, supraphysiological overexpression of BCL11B in human HSPC accelerates their differentiation into mature functional T-cells and increases the output of mature T-cells in an in vitro T-cell differentiation model. Furthermore, the mature T-cells produced from BCL11B overexpressing HSPC have enhanced function and delayed exhaustion compared to T-cells produced from control non-overexpressing HSPC.

Accordingly, BCL11B expression in host cells (such as T-cells) can be used at least for: 1) enhancing and expediting thymic T-cell reconstitution post bone marrow transplantation; 2) enhancing the function and persistence and prevent the exhaustion of engineered T-cells (such as CAR T cells) that are infused into patients as immunotherapies for cancer; and 3) generating adequate output of functional T-cells from pluripotent stem cells for the ex vivo generation of allogenic off the shelf immunotherapy T-cell products for patients (for the third application, BCL11B activation will be used in concert with an ex vivo culture system for generating T-cells from pluripotent stem cells).

Published studies of murine multi-lineage or T-cell progenitors with a homozygous deletion of the transcription factor gene BCL11B have shown that BCL11B is required for normal T-cell differentiation and function in mice. However, BCL11B is not required for initiation of T-cell gene expression in murine HSPC (Li et al, Science 2010) and unexpectedly, BCL11B gain of function by overexpression leads to cell death in murine HSPC. Furthermore, until now, evidence showing that supraphysiological activation of BCL11B enhances or accelerates differentiation of human or murine hematopoietic progenitor cells into mature T cells or improves the function of T-cells has not been reported.

Other transcription factors critical for the initial stages of thymopoiesis include TCF7, GATA3, and NOTCH1. Gain of function of Tcf7 and Gata3 have not been reported to enhance differentiation of murine HSPC into SP T-cells. Moreover, TCF7 or GATA3 overexpression do not increase the generation of SP TCRαβ+ T-cells from human CB HSPC (Van de Walle et al., Nat Commun. 2016; 7:11171). Of note, while knockdown of GATA3 or inhibition of NOTCH1 signaling impairs or abrogates T-cell differentiation of human thymic progenitors respectively (Van de Walle et al., Nat Commun. 2016; 7:11171; Van de Walle et al., J Exp Med. 2013 Apr. 8; 210(4):683-97), gain of function of these genes inhibits the generation of TCRαβ+ cells (Van de Walle et al., J Exp Med. 2013 Apr. 8; 210(4):683-97; Taghon et al., J Immunol. 2001 Oct. 15; 167(8):4468-75). A non-limiting explanation is that the need for precise, stage-specific regulation of the timing of expression of these genes during thymopoiesis may account for the paradoxical effects on T-cell differentiation when these genes are overexpressed. These results highlight the unpredictability associated with gain- and loss-of function studies of transcription factor function, and show that gain of function results are not predictable from loss of function studies, particularly in the context of T-cell differentiation.

### II. Terms

Unless otherwise noted, technical terms are used according to conventional usage. Definitions of common terms in molecular biology may be found in Benjamin Lewin, Genes X, published by Jones & Bartlett Publishers, 2009; and Meyers et al. (eds.), The Encyclopedia of Cell Biology and Molecular Medicine, published by Wiley-VCH in 16 volumes, 2008; and other similar references.

As used herein, the singular forms “a,” “an,” and “the,” refer to both the singular as well as plural, unless the context clearly indicates otherwise. For example, the term “a cell” includes single or plural cells and can be considered equivalent to the phrase “at least one cell.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting.

In order to facilitate review of the various embodiments of this disclosure, the following explanations of specific terms are provided:

About: Unless context indicated otherwise, “about” refers to plus or minus 5% of a reference value. For example, “about” 100 refers to 95 to 105.

Administration: The introduction of a composition into a subject by a chosen route. Administration can be local or systemic. For example, if the chosen route is intravenous, the composition is administered by introducing the composition into a vein of the subject. Exemplary routes of administration include, but are not limited to, oral, injection (such as subcutaneous, intramuscular, intradermal, intraperitoneal, intra-articular, intrathecal (such as lumbar puncture) and intravenous), sublingual, rectal, transdermal (for example, topical), intranasal, vaginal, and inhalation routes.

Autoimmune disorder: A disorder in which the immune system produces an immune response (for example, a B cell or a T cell response) against an endogenous antigen, with consequent injury to tissues. For example, rheumatoid arthritis is an autoimmune disorder, as are Hashimoto's thyroiditis, pernicious anemia, Addison's disease, type I diabetes, systemic lupus erythematosus, dermatomyositis, Sjogren's syndrome, dermatomyositis, lupus erythematosus, multiple sclerosis, myasthenia gravis, Reiter's syndrome, graft-vs-host disease, and Grave's disease, among others.

B-cell lymphoma/leukemia 11B protein (BCL11B): A protein that in humans is encoded by the BCL11B gene. Non-limiting examples of BCL11B protein sequence can be found in GenBank No. NP_612808.1, NP_075049.1, NP_001269167.1, and NP_001269166.1, each of which is incorporated by reference herein.

CD34: A cell surface antigen formerly known as hematopoietic progenitor cell antigen 1, and MY10, is a known marker of human hematopoietic stem cells. The human CD34 gene, which maps to chromosome 1q32, spans 26 kb and has 8 exons. CD34 is a 67 kDa transmembrane glycoprotein. CD34 is expressed selectively on human hematopoietic progenitor cells. The biological function of CD34 is still unknown.

Chimeric Antigen Receptor (CAR): An engineered T cell receptor having an extracellular antibody-derived targeting domain (such as an scFv) joined to one or more intracellular signaling domains of a T cell receptor. A “chimeric antigen receptor T cell” is a T cell expressing a CAR, and has antigen specificity determined by the antibody-derived targeting domain of the CAR. Methods of making CARs are available (see, e.g., Park et al., Trends Biotechnol., 29:550-557, 2011; Grupp et al., N Engl J Med., 368:1509-1518, 2013; Han et al., J. Hematol Oncol., 6:47, 2013; PCT Pubs. WO2012/079000, WO2013/059593; and U.S. Pub. 2012/0213783, each of which is incorporated by reference herein in its entirety.)

Control: A reference standard. In some embodiments, the control is a negative control, such as cell or cell population that has not been modified to have increased expression of BCL11B. In other embodiments, the control is a positive control, such as a cell with a known level of BCL11B expression. In still other embodiments, the control is a historical control or standard reference value or range of values (such as a previously tested control sample, such as a group of patients with known prognosis or outcome, or group of samples that represent baseline or normal values).

A difference between a test sample and a control can be an increase or conversely a decrease. The difference can be a qualitative difference or a quantitative difference, for example a statistically significant difference. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, or at least about 500%.

Expression: Transcription or translation of a nucleic acid sequence. For example, a gene can be expressed when its DNA is transcribed into an RNA or RNA fragment, which in some examples is processed to become mRNA. A gene may also be expressed when its mRNA is translated into an amino acid sequence, such as a protein or a protein fragment. In a particular example, a heterologous gene is expressed when it is transcribed into an RNA. In another example, a heterologous gene is expressed when its RNA is translated into an amino acid sequence. Regulation of expression can include controls on transcription, translation, RNA transport and processing, degradation of intermediary molecules such as mRNA, or through activation, inactivation, compartmentalization or degradation of specific protein molecules after they are produced.

Expression Control Sequences: Nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcription terminators, a start codon (ATG) in front of a protein-encoding gene, splicing signal for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

A promoter is a minimal sequence sufficient to direct transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included. For example, when cloning in bacterial systems, inducible promoters such as pL of bacteriophage lambda, plac, ptrp, ptac (ptrp-lac hybrid promoter) and the like may be used. In one embodiment, when cloning in mammalian cell systems, promoters derived from the genome of mammalian cells (such as metallothionein promoter) or from mammalian viruses (such as the retrovirus long terminal repeat; the adenovirus late promoter; the vaccinia virus 7.5K promoter) can be used. Promoters produced by recombinant DNA or synthetic techniques may also be used to provide for transcription of the nucleic acid sequences.

A polynucleotide can be inserted into an expression vector that contains a promoter sequence, which facilitates the efficient transcription of the inserted genetic sequence of the host. The expression vector typically contains an origin of replication, a promoter, as well as specific nucleic acid sequences that allow phenotypic selection of the transformed cells.

Expression vector: A vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

Hematopoietic Stem and Progenitor Cell (HSPCs): Hematopoietic stem cell is a multipotent and self renewing cell that gives rise to progeny in all defined hematolymphoid lineages. In addition, limiting numbers of HSPC are capable of fully reconstituting an immunocompromised subject in all blood cell types and their progenitors, including the hematopoietic stem cell, by cell renewal. A “progenitor cell” is a non-self renewing cell that gives rise to progeny in a defined cell lineage (unilineage progenitor) or multiple cell lineages (multilineage progenitor). One specific non-limiting example of a hematopoietic stem and progenitor cell is a “T cell progenitor cell,” which gives rise to immature and mature T cells. Non-limiting markers for HSPCs include CD34.

Heterologous: Originating from a different genetic source. A nucleic acid molecule that is heterologous to a cell originated from a genetic source other than the cell in which it is expressed. In one specific, non-limiting example, a heterologous nucleic acid molecule encoding a protein, such as BCL11B, is expressed in a cell, such as a mammalian cell. Methods for introducing a heterologous nucleic acid molecule in a cell or organism are well known in the art, for example transformation with a nucleic acid, including electroporation, lipofection, particle gun acceleration, and homologous recombination.

Neoplasia, cancer, or tumor: A neoplasm is an abnormal growth of tissue or cells that results from exces-sive cell division. Neoplastic growth can produce a tumor. The amount of a tumor in an individual is the “tumor burden” which can be measured as the number, volume, or weight of the tumor. A tumor that does not metastasize is referred to as “benign.” A tumor that invades the surround-ing tissue or can metastasize (or both) is referred to as “malignant.”

Tumors of the same tissue type are primary tumors originating in a particular organ and may be divided into tumors of different sub-types. For examples, lung carcinomas can be divided into an adenocarcinoma, small cell, squamous cell, or non-small cell tumors.

Examples of solid tumors, such as sarcomas (connective tissue cancer) and carcinomas (epithelial cell cancer), include fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colorectal carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, and CNS tumors (such as a glioma, astrocytoma, glioblastoma, medulloblastoma, craniopharyogioma, ependymoma, pinealoma, hemangioblastoma, acoustic neuroma, oligodendroglioma, menangioma, melanoma, neuroblastoma and retinoblastoma).

Examples of hematological or lymphoid cancers include leukemias, for example acute leukemias (such as acute lymphoblastic leukemia (such as T-ALL or B-ALL), acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), a polycythemia vera, a lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Nucleic acid molecule: A polymeric form of nucleotides, which may include both sense and anti-sense strands of RNA, cDNA, genomic DNA, and synthetic forms and mixed polymers of the above. A nucleotide refers to a ribonucleotide, deoxynucleotide or a modified form of either type of nucleotide. The term “nucleic acid molecule” as used herein is synonymous with “nucleic acid” and “polynucleotide.” A nucleic acid molecule is usually at least 10 bases in length, unless otherwise specified. The term includes single- and double-stranded forms of DNA. A polynucleotide may include either or both naturally occurring and modified nucleotides linked together by naturally occurring and/or non-naturally occurring nucleotide linkages. “cDNA” refers to a DNA that is complementary or identical to an mRNA, in either single stranded or double stranded form. “Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom.

Operably linked: A first nucleic acid sequence is operably linked with a second nucleic acid sequence when the first nucleic acid sequence is placed in a functional relationship with the second nucleic acid sequence. For instance, a promoter, such as the MND promoter, is operably linked to a coding sequence if the promoter affects the transcription or expression of the coding sequence. Generally, operably linked DNA sequences are contiguous and, where necessary to join two protein-coding regions, in the same reading frame.

Pluripotent Stem Cell: A cell that has the capacity to self-renew indefinitely by dividing and is pluripotent, and as such has the capacity to develop into any one of the three primary germ cell layers (e.g. cells of the ectoderm, endoderm, and mesoderm), and therefore into any cell lineage in the body. Pluripotent stem cells include, but are not limited to, embryonic stem cells and induced pluripotent stem cells. Non-limiting markers for pluripotent stem cells include CD326+ (EpCAM+).

Subject: Living multi-cellular vertebrate organisms, a category that includes human and non-human mammals. In an example, a subject is a human.

T Cell: A white blood cell critical to the immune response. T cells include, but are not limited to, CD4+ T cells and CD8+ T cells. A CD4+ T lymphocyte is an immune cell that carries a marker on its surface known as “cluster of differentiation 4” (CD4). These cells, also known as helper T cells, help orchestrate the immune response, including antibody responses as well as killer T cell responses. In another embodiment, a CD4+ cell is a regulatory T cell (Treg). CD8+ T cells carry the “cluster of differentiation 8” (CD8) marker. In one embodiment, a CD8 T cell is a cytotoxic T lymphocyte. An effector function of a T cell is a specialized function of the T cell, such as cytolytic activity or helper activity including the secretion of cytokines. A mature T cell is a T cell that is CD3+CD4+CD8− or CD3+CD4−CD8+.

T Cell Therapy: A therapeutic intervention that includes administering T cells to a subject, or administering cells that will mature into T cells to the subject. Non-limiting examples of T cell therapies include administration of HSPC for thymic T cell reconstitution in a subject, and administration of a CAR T cell therapy or an engineered T cell receptor (TCR) T cell therapy for treatment of cancer in a subject.

Therapeutically effective amount: A quantity of a therapeutic sufficient to achieve a desired effect in a subject to whom the therapeutic is administered, such as for treatment. In a non-limiting example, this can be an amount of mature T cells with increased BCL11B expression as described herein that improves T cell reconstitution in a HSCT patient following the transplant. Ideally, a therapeutically effective amount provides a therapeutic effect without causing a substantial cytotoxic effect in the subject.

The therapeutically effective amount of a therapeutic that is administered to a subject will vary depending upon a number of factors associated with that subject, for example the overall health and/or weight of the subject, the severity and type of the condition being treated, and/or the manner of administration. A therapeutically effective amount encompasses a fractional dose that contributes in combination with previous or subsequent administrations to attaining an effective response. For example, a therapeutically effective amount of modified cells with increased BCL11B expression as described herein can be administered in a single dose (or infusion), or in several doses, for example daily, during a course of treatment lasting several days or weeks. A therapeutically effective amount can be determined by varying the dosage and measuring the resulting therapeutic response, such as improved T cell reconstitution.

Transduced: A transduced cell is a cell into which a nucleic acid molecule has been introduced by molecular biology techniques. As used herein, the term transduced and the like (e.g., transformation, transfection, transduction, transformed, etc.) encompasses all techniques by which a nucleic acid molecule might be introduced into such a cell, including transduction with viral vectors, transformation with plasmid vectors, and introduction of DNA by electroporation, lipofection, and particle gun acceleration.

Treating, Inhibiting, or Preventing a Disease or Condition: “Preventing” a disease or condition refers to inhibiting the full development of a disease or condition. “Treating” refers to a therapeutic intervention that ameliorates a sign or symptom of a disease or condition after it has begun to develop, such as a reduction in tumor burden or a decrease in the number of size of metastases. “Ameliorating” refers to the reduction in the number or severity of signs or symptoms of a disease or condition, such as cancer.

Vector: A nucleic acid molecule as introduced into a host cell, thereby producing a transformed host cell. Recombinant DNA vectors are vectors having recombinant DNA. A vector can include nucleic acid sequences that permit it to replicate in a host cell, such as an origin of replication. A vector can also include one or more selectable marker genes and other genetic elements known in the art. Viral vectors are recombinant nucleic acid vectors having at least some nucleic acid sequences derived from one or more viruses. A replication deficient viral vector is a vector that requires complementation of one or more regions of the viral genome required for replication due to a deficiency in at least one replication-essential gene function. For example, such that the viral vector does not replicate in typical host cells, especially those in a human patient that could be infected by the viral vector in the course of a therapeutic method.

Under conditions sufficient for: A phrase that is used to describe any environment that permits a desired activity.

III. Modified Cells with Increased BCL11B Expression

The methods disclosed herein utilize HSPCs, pluripotent stem cells, T cells (such as mature T cells), or combinations thereof, that are modified to have increased BCL11B expression. The increase in BCL11B expression is accomplished by any suitable means, such as transducing the HSPCs, pluripotent stem cells, or mature T cells with a vector (such as a lentiviral vector) encoding BCL11B operably linked to a promoter. In some embodiments, the BCL11B gene is inserted, using gene editing technology, such as CRISPR/Cas9 or TALEN, into an area of the genome that allows increased and/or regulated expression of BCL11B, such as from an endogenous promoter.

In any embodiment described herein using pluripotent stem cells, cells derived from the pluripotent stem cells, such as a mesodermal progenitor cell or any cell derived from a pluripotent stem cell that is capable of maturing to a T cell, can be used in place of the pluripotent stem cells.

In another embodiment, the increase in BCL11B expression is accomplished by treating the HSPCs, pluripotent stem cells, or mature T cells with an agent that targets the promoter of the native BCL11B gene to increase its expression in the cell, such as by using CRISPR/Cas9 technology. The increase in BCL11B expression in the modified cells increases production and/or proliferation of T cells from the HSPCs or the pluripotent stem cells, or increases proliferation of the mature T cells, compared to the corresponding control cells without the increase in BCL11B expression. In several embodiments, the modified cells are administered to a subject in need thereof.

The B-cell lymphoma/leukemia 11B protein (BCL11B) in humans is encoded by the BCL11B gene. Without being bound by theory, BCL11B is one of multiple transcription factors, including TCF7, NOTCH1, and GATA3, involved in thymopoeisis in humans. In some embodiments, increasing BCL11B expression accelerates thymopoiesis of HSPCs and pluripotent stem cells and increases production of T cells, such as mature T cells, from HSPCs or pluripotent stem cells. Additionally, increasing BCL11B expression in mature T cells increases proliferation of the mature T cells. In certain embodiments, T cells proliferating from the modified cells have delayed exhaustion, an increased central memory immunophenotype, and/or increased interleukin 2 production and/or TNF-alpha production compared to corresponding control cells. In certain disclosed embodiments, the T cells with an enhanced central memory immunophenotype may be CD45RO+CD62L+CCR7+ T cells. T cell production and/or proliferation from the modified cells may be independent of Notch signaling.

In some embodiments, increasing BCL11B expression includes transforming the cells with a heterologous nucleic acid encoding BCL11B. In some embodiments, the cells are transduced with a vector encoding BCL11B. In a specific non-limiting example, the vector is a lentiviral vector.

Exemplary nucleic acid sequences encoding human BCL11B are set forth in GENBANK Accession Nos. NM_138576.4 and NM_022898.3, which are incorporated by reference herein.

An exemplary nucleic acid sequence encoding BCL11B isoform 1 protein is set forth as:

(SEQ ID NO: 2)
atgtcccgccgcaaacagggcaacccgcagcacttgtcccagagggagct
catcaccccagaggctgaccatgtggaggccgccatcctcgaagaagacg
agggtctggagatagaggagccaagtggcctggggctgatggtgggtggc
cccgaccctgacctgctcacctgtggccagtgtcaaatgaacttcccctt
gggggacatcctggtttttatagagcacaaaaggaagcagtgtggcggca
gcttgggtgcctgctatgacaaggccctggacaaggacagcccgccaccc
tcctcacgctccgagctcaggaaagtgtccgagccggtggagatcgggat
ccaagtcacccccgacgaagatgaccacctgctctcacccacgaaaggca
tctgtcccaagcaggagaacattgcagggccgtgcaggcctgcccagctg
ccagcggtggcccccatagctgcctcctcccaccctcactcatccgtgat
cacttcacctctgcgtgccctgggcgctctcccgccctgcctccccctgc
cgtgctgcagcgcgcgcccggtctcgggtgacgggactcagggtgagggt
cagacggaggctccctttggatgccagtgtcagttgtcaggtaaagatga
gccttccagctacatttgcacaacatgcaagcagcccttcaacagcgcgt
ggttcctgctgcagcacgcgcagaacacgcacggcttccgcatctacctg
gagcccgggccggccagcagctcgctcacgccgcggctcaccatcccgcc
gccgctcgggccggaggccgtggcgcagtccccgctcatgaatttcctgg
gcgacagcaaccccttcaacctgctgcgcatgacgggccccatcctgcgg
gaccacccgggcttcggcgagggccgcctgccgggcacgccgcctctctt
cagtcccccgccgcgccaccacctggacccgcaccgcctcagtgccgagg
agatggggctcgtcgcccagcaccccagtgccttcgaccgagtcatgcgc
ctgaaccccatggccatcgactcgcccgccatggacttctcgcggcggct
ccgcgagctggcgggcaacagctccacgccgccgcccgtgtccccgggcc
gcggcaaccctatgcaccggctcctgaaccccttccagcccagccccaag
tccccgttcctgagcacgccgccgctgccgcccatgccccctggcggcac
gccgcccccgcagccgccagccaagagcaagtcgtgcgagttctgcggca
agaccttcaagttccagagcaatctcatcgtgcaccggcgcagtcacacg
ggcgagaagccctacaagtgccagctgtgcgaccacgcgtgctcgcaggc
cagcaagctcaagcgccacatgaagacgcacatgcacaaggccggctcgc
tggccggccgctccgacgacgggctctcggccgccagctcccccgagccc
ggcaccagcgagctggcgggcgagggcctcaaggcggccgacggtgactt
ccgccaccacgagagcgacccgtcgctgggccacgagccggaggaggagg
acgaggaggaggaggaggaggaggaggagctgctactggagaacgagagc
cggcccgagtcgagcttcagcatggactcggagctgagccgcaaccgcga
gaacggcggtggtggggtgcccggggtcccgggcgcggggggcggcgcgg
ccaaggcgctggctgacgagaaggcgctggtgctgggcaaggtcatggag
aacgtgggcctaggcgcactgccgcagtacggcgagctcctggccgacaa
gcagaagcgcggcgccttcctgaagcgtgcggcgggcggcggggacgcgg
gcgacgacgacgacgcgggcggctgcggggacgcgggcgcgggcggcgcg
gtcaacgggcgcgggggcggcttcgcgccaggcaccgagcccttccccgg
gctcttcccgcgcaagcccgcgccgctgcccagccccgggctcaacagcg
ccgccaagcgcatcaaggtggagaaggacctggagctgccgcccgccgcg
ctcatcccgtccgagaacgtgtactcgcagtggctggtgggctacgcggc
gtcgcggcacttcatgaaggaccccttcctgggcttcacggacgcacgac
agtcgcccttcgccacgtcgtccgagcactcgtccgagaacggcagcctg
cgcttctccacgccgcccggggacctgctggacggcggcctctcgggccg
cagcggcacggccagcggaggcagcaccccgcacctgggcggcccgggcc
ccgggcggcccagctccaaggagggccgccgcagcgacacgtgcgagtac
tgcggcaaggtgttcaagaactgcagcaacttgacggtgcaccggcggag
ccacaccggcgagcggccttacaagtgcgagctgtgcaactacgcgtgcg
cgcagagcagcaagctcacgcgccacatgaagacgcacgggcagatcggc
aaggaggtgtaccgctgcgacatctgccagatgcccttcagcgtctacag
caccctggagaaacacatgaaaaagtggcacggcgagcacttgctgacta
acgacgtcaaaatcgagcaggccgagaggagctaa

An exemplary nucleic acid sequence encoding BCL11B isoform 2 protein is set forth as:

(SEQ ID NO: 4)
atgtcccgccgcaaacagggcaacccgcagcacttgtcccagagggagct
catcaccccagaggctgaccatgtggaggccgccatcctcgaagaagacg
agggtctggagatagaggagccaagtggcctggggctgatggtgggtggc
cccgaccctgacctgctcacctgtggccagtgtcaaatgaacttcccctt
gggggacatcctggtttttatagagcacaaaaggaagcagtgtggcggca
gcttgggtgcctgctatgacaaggccctggacaaggacagcccgccaccc
tcctcacgctccgagctcaggaaagtgtccgagccggtggagatcgggat
ccaagtcacccccgacgaagatgaccacctgctctcacccacgaaaggca
tctgtcccaagcaggagaacattgcaggtaaagatgagccttccagctac
atttgcacaacatgcaagcagcccttcaacagcgcgtggttcctgctgca
gcacgcgcagaacacgcacggcttccgcatctacctggagcccgggccgg
ccagcagctcgctcacgccgcggctcaccatcccgccgccgctcgggccg
gaggccgtggcgcagtccccgctcatgaatttcctgggcgacagcaaccc
cttcaacctgctgcgcatgacgggccccatcctgcgggaccacccgggct
tcggcgagggccgcctgccgggcacgccgcctctcttcagtcccccgccg
cgccaccacctggacccgcaccgcctcagtgccgaggagatggggctcgt
cgcccagcaccccagtgccttcgaccgagtcatgcgcctgaaccccatgg
ccatcgactcgcccgccatggacttctcgcggcggctccgcgagctggcg
ggcaacagctccacgccgccgcccgtgtccccgggccgcggcaaccctat
gcaccggctcctgaaccccttccagcccagccccaagtccccgttcctga
gcacgccgccgctgccgcccatgccccctggcggcacgccgcccccgcag
ccgccagccaagagcaagtcgtgcgagttctgcggcaagaccttcaagtt
ccagagcaatctcatcgtgcaccggcgcagtcacacgggcgagaagccct
acaagtgccagctgtgcgaccacgcgtgctcgcaggccagcaagctcaag
cgccacatgaagacgcacatgcacaaggccggctcgctggccggccgctc
cgacgacgggctctcggccgccagctcccccgagcccggcaccagcgagc
tggcgggcgagggcctcaaggcggccgacggtgacttccgccaccacgag
agcgacccgtcgctgggccacgagccggaggaggaggacgaggaggagga
ggaggaggaggaggagctgctactggagaacgagagccggcccgagtcga
gcttcagcatggactcggagctgagccgcaaccgcgagaacggcggtggt
ggggtgcccggggtcccgggcgcggggggcggcgcggccaaggcgctggc
tgacgagaaggcgctggtgctgggcaaggtcatggagaacgtgggcctag
gcgcactgccgcagtacggcgagctcctggccgacaagcagaagcgcggc
gccttcctgaagcgtgcggcgggcggcggggacgcgggcgacgacgacga
cgcgggcggctgcggggacgcgggcgcgggcggcgcggtcaacgggcgcg
ggggcggcttcgcgccaggcaccgagcccttccccgggctcttcccgcgc
aagcccgcgccgctgcccagccccgggctcaacagcgccgccaagcgcat
caaggtggagaaggacctggagctgccgcccgccgcgctcatcccgtccg
agaacgtgtactcgcagtggctggtgggctacgcggcgtcgcggcacttc
atgaaggaccccttcctgggcttcacggacgcacgacagtcgcccttcgc
cacgtcgtccgagcactcgtccgagaacggcagcctgcgcttctccacgc
cgcccggggacctgctggacggcggcctctcgggccgcagcggcacggcc
agcggaggcagcaccccgcacctgggcggcccgggccccgggcggcccag
ctccaaggagggccgccgcagcgacacgtgcgagtactgcggcaaggtgt
tcaagaactgcagcaacttgacggtgcaccggcggagccacaccggcgag
cggccttacaagtgcgagctgtgcaactacgcgtgcgcgcagagcagcaa
gctcacgcgccacatgaagacgcacgggcagatcggcaaggaggtgtacc
gctgcgacatctgccagatgcccttcagcgtctacagcaccctggagaaa
cacatgaaaaagtggcacggcgagcacttgctgactaacgacgtcaaaat
cgagcaggccgagaggagctaa

In some embodiments, the nucleic acid sequence encoding BCL11B is at least 90%, at least 95%, at least 99%, or 100% identical to any one of SEQ ID NOs: 2 or 4.

Exemplary human BCL11B proteins are set forth as GENBANK Accession Nos. NP_612808.1 and NP_075049.1, which are incorporated herein by reference.

The amino acid sequence of human BCL11B isoform 1 is set forth as:

(SEQ ID NO: 1)
MSRRKQGNPQHLSQRELITPEADHVEAAILEEDEGLEIEEPSGLGLMVGG
PDPDLLTCGQCQMNFPLGDILVFIEHKRKQCGGSLGACYDKALDKDSPPP
SSRSELRKVSEPVEIGIQVTPDEDDHLLSPTKGICPKQENIAGPCRPAQL
PAVAPIAASSHPHSSVITSPLRALGALPPCLPLPCCSARPVSGDGTQGEG
QTEAPFGCQCQLSGKDEPSSYICTTCKQPFNSAWFLLQHAQNTHGFRIYL
EPGPASSSLTPRLTIPPPLGPEAVAQSPLMNFLGDSNPFNLLRMTGPILR
DHPGFGEGRLPGTPPLFSPPPRHHLDPHRLSAEEMGLVAQHPSAFDRVMR
LNPMAIDSPAMDFSRRLRELAGNSSTPPPVSPGRGNPMHRLLNPFQPSPK
SPFLSTPPLPPMPPGGTPPPQPPAKSKSCEFCGKTFKFQSNLIVHRRSHT
GEKPYKCQLCDHACSQASKLKRHMKTHMHKAGSLAGRSDDGLSAASSPEP
GTSELAGEGLKAADGDFRHHESDPSLGHEPEEEDEEEEEEEEELLLENES
RPESSFSMDSELSRNRENGGGGVPGVPGAGGGAAKALADEKALVLGKVME
NVGLGALPQYGELLADKQKRGAFLKRAAGGGDAGDDDDAGGCGDAGAGGA
VNGRGGGFAPGTEPFPGLFPRKPAPLPSPGLNSAAKRIKVEKDLELPPAA
LIPSENVYSQWLVGYAASRHFMKDPFLGFTDARQSPFATSSEHSSENGSL
RFSTPPGDLLDGGLSGRSGTASGGSTPHLGGPGPGRPSSKEGRRSDTCEY
CGKVFKNCSNLTVHRRSHTGERPYKCELCNYACAQSSKLTRHMKTHGQIG
KEVYRCDICQMPFSVYSTLEKHMKKWHGEHLLTNDVKIEQAERS

The amino acid sequence of human BCL11B isoform 2 is set forth as:

(SEQ ID NO: 5)
MSRRKQGNPQHLSQRELITPEADHVEAAILEEDEGLEIEEPSGLGLMVGG
PDPDLLTCGQCQMNFPLGDILVFIEHKRKQCGGSLGACYDKALDKDSPPP
SSRSELRKVSEPVEIGIQVTPDEDDHLLSPTKGICPKQENIAGKDEPSSY
ICTTCKQPFNSAWFLLQHAQNTHGFRIYLEPGPASSSLTPRLTIPPPLGP
EAVAQSPLMNFLGDSNPFNLLRMTGPILRDHPGFGEGRLPGTPPLFSPPP
RHHLDPHRLSAEEMGLVAQHPSAFDRVMRLNPMAIDSPAMDFSRRLRELA
GNSSTPPPVSPGRGNPMHRLLNPFQPSPKSPFLSTPPLPPMPPGGTPPPQ
PPAKSKSCEFCGKTFKFQSNLIVHRRSHTGEKPYKCQLCDHACSQASKLK
RHMKTHMHKAGSLAGRSDDGLSAASSPEPGTSELAGEGLKAADGDFRHHE
SDPSLGHEPEEEDEEEEEEEEELLLENESRPESSFSMDSELSRNRENGGG
GVPGVPGAGGGAAKALADEKALVLGKVMENVGLGALPQYGELLADKQKRG
AFLKRAAGGGDAGDDDDAGGCGDAGAGGAVNGRGGGFAPGTEPFPGLFPR
KPAPLPSPGLNSAAKRIKVEKDLELPPAALIPSENVYSQWLVGYAASRHF
MKDPFLGFTDARQSPFATSSEHSSENGSLRFSTPPGDLLDGGLSGRSGTA
SGGSTPHLGGPGPGRPSSKEGRRSDTCEYCGKVFKNCSNLTVHRRSHTGE
RPYKCELCNYACAQSSKLTRHMKTHGQIGKEVYRCDICQMPFSVYSTLEK
HMKKWHGEHLLTNDVKIEQAERS

In some embodiments, the BCL11B protein expressed in the HSPCs, pluripotent stem cells, or mature T cells comprises an amino acid sequence at least 90%, at least 95%, at least 99%, or 100% identical to any one of SEQ ID NOs: 1 or 5.

The nucleic acid encoding BCL11B is typically operably linked to a heterologous promoter. The promoter is selected such that the transduced HSPCs, pluripotent stem cells, or mature T cells produce a sufficient increase in BCL11B expression to increase production and/or proliferation of T cells from the HSPCs or the pluripotent stem cells, or increase proliferation of the mature T cells, compared to corresponding control cells without the increase in BCL11B expression.

The promoter can be any suitable promotor, including constitutive and inducible promoters. In some embodiments, the promoter in a non-viral promoter, in other embodiments, the promoter is a viral promoter. Any promoter can be used that provides a sufficient expression level of BCL11B when operably linked to a nucleic acid sequence encoding BCL11B and introduced into the HSPCs, pluripotent stem cells, or mature T cells. The promoter can be, for example, a myeloproliferative sarcoma virus enhancer, negative control region deleted, d1587rev primer-binding site substituted (MND) promoter, a Murine Embryonic Stem Cell Virus (MSCV) promoter, a Phosphoglycerate Kinase-1 (PGK) promoter, a beta-globin, human cytomegalovirus (CMV) promoter, a human elongation factor-1 alpha (EF1alpha) promoter. In one non-limiting embodiment the HSPCs, pluripotent stem cells, or mature T cells, are transduced with a lentiviral vector including a nucleic acid encoding BLC11b that is operably linked to a MND promoter.

A non-limiting example of a sequence for a promoter that can be used with the disclosed embodiments is provided below:

MND promoter
(SEQ ID NO: 3)
ATCGATTAGTCCAATTTGTTAAAGACAGGATATCAGTGGTCCAGGCTCTA
GTTTTGACTCAACAATATCACCAGCTGAAGCCTATAGAGTACGAGCCATA
GATAGAATAAAAGATTTTATTTAGTCTCCAGAAAAAGGGGGGAATGAAAG
ACCCCACCTGTAGGTTTGGCAAGCTAGGATCAAGGTTAGGAACAGAGAGA
CAGCAGAATATGGGCCAAACAGGATATCTGTGGTAAGCAGTTCCTGCCCC
GGCTCAGGGCCAAGAACAGTTGGAACAGCAGAATATGGGCCAAACAGGAT
ATCTGTGGTAAGCAGTTCCTGCCCCGGCTCAGGGCCAAGAACAGATGGTC
CCCAGATGCGGTCCCGCCCTCAGCAGTTTCTAGAGAACCATCAGATGTTT
CCAGGGTGCCCCAAGGACCTGAAATGACCCTGTGCCTTATTTGAACTAAC
CAATCAGTTCGCTTCTCGCTTCTGTTCGCGCGCTTCTGCTCCCCGAGCTC
AATAAAAGAGCCCACAACCCCTCACTCGGCGCGATC

Additional information regarding exemplary promoters that can be used in the disclosed embodiments can be found in Halene et al., Improved expression in hematopoietic and lymphoid cells in mice after transplantation of bone marrow transduced with a modified retroviral vector, Blood, 1999, 94:3349-3357, which is disclosed by reference herein in its entirety.

Polynucleotide sequences encoding BCL11B, can be inserted into an expression vector, such as a plasmid, virus or other vehicle known in the art that has been manipulated by insertion or incorporation of the BCL11B sequence. Polynucleotide sequences which encode BCL11B can be operatively linked to the promoter and optionally additional expression control sequences. In one embodiment, an expression control sequence operatively linked to a coding sequence is ligated such that expression of the coding sequence is achieved under conditions compatible with the expression control sequences. The expression vector typically contains an origin of replication, a promoter, and specific genes that allow phenotypic selection of the transformed cells. Optionally, the expression vector can encode other molecules, such as, but not limited to, a chimeric antigen receptor or an engineered T cell receptor.

An expression vector can optionally include a suicide gene, such as HSV thymidine kinase (HSV-TK). In such embodiments, once the T cell therapy is complete, the majority of genetically engineered cells can be killed off by administration of ganciclovir (GCV). HSV-TK converts GCV into a toxic product and allows selective elimination of TK+ cells. An exemplary working concentration of GCV is 10-100 mg/kg/day for 7-21 days.

In one example, the vector is a viral vector, such as a retroviral vector, an adenoviral vector, or an adeno-associated vector (AAV). In specific, non-limiting examples, the retroviral vector is a lentiviral vector.

Examples of retroviral vectors in which a foreign gene can be inserted include, but are not limited to: Moloney murine leukemia virus (MoMLV), Harvey murine sarcoma virus (HaMuSV), murine mammary tumor virus (MuMTV), and Rous Sarcoma Virus (RSV). In one embodiment, when the subject is a human, a vector such as the gibbon ape leukemia virus (GALV) can be utilized. In some embodiments, the retroviral vector is a derivative of a murine or avian retrovirus, or a human or primate lentivirus.

The retrovirus genome includes two LTRs, an encapsidation sequence and three coding regions (gag, pol and env). In recombinant retrovirus vectors, the gag, pol and env genes are generally deleted, in whole or in part, and replaced with a heterologous nucleic acid sequence of interest, such as a nucleic acid sequence encoding BCL11B operably linked to a promoter.

Since recombinant retroviruses are typically defective, they require assistance in order to produce infectious vector particles. This assistance can be provided, for example, by using helper cell lines that contain plasmids encoding all of the structural genes of the retrovirus under the control of regulatory sequences within the long terminal repeat (LTR). These plasmids are missing a nucleotide sequence which enables the packaging mechanism to recognize an RNA transcript for encapsidation. Helper cell lines which have deletions of the packaging signal include, but are not limited to ψ2, PA317, and PA12, for example. Alternatively, NIH 3T3 or other tissue culture cells can be directly transfected with plasmids encoding the retroviral structural genes gag, pol and env, by conventional calcium phosphate transfection. These cell lines produce empty virions, since no genome is packaged. If a retroviral vector is introduced into such cells in which the packaging signal is intact, but the structural genes are replaced by other genes of interest, the vector can be packaged and vector virion produced. These cells are then transfected with the vector plasmid containing the genes of interest. The resulting cells release the retroviral vector into the culture medium. Thus, for production of viral particles, the gag, pol and env genes are coexpressed in the packaging cell line.

In additional embodiments, the nucleic acid molecule encoding BCL11B is targeted into a specific site in the genome of the HSPC, pluripotent stem cell, or mature T cell using clustered, regularly interspaced, short palindromic repeat (CRISPR) technology. This approach generates RNA-guided nucleases, such as Cas9, with customizable specificities. The CRISPR/Cas system can be used for gene editing (adding, disrupting or changing the sequence of specific genes) and gene regulation in species throughout the tree of life (Mali et al., Nature Methods 10:957-963, 2013). By delivering the Cas9 protein and appropriate guide RNAs into a cell, the organism's genome can be cut at any desired location and the heterologous nucleic acid encoding BCL11B operably linked to a promoter inserted at the site.

In some embodiments, the nucleic acid encoding BCL11B operably linked to a promoter is targeted into a specific site in the nuclei of the HSPC, pluripotent stem cell, or mature T cell using transcription activator-like effector nuclease (TALEN) technology. Methods are available for designing TALENs for targeting particular genomic sties (see, for example, Bogdanove and Voytas, Science. 2011 Sep. 30; 333(6051):1843-6). TALEN-mediated gene targeting is effective in stem cells and mature T cells. Genomic editing with TALENs capitalizes on the cell's ability to undergo homology directed repair (HDR), following an induced and targeted double-stranded DNA break (DSB). During this time a donor DNA template can be provided to the cell to insert new transgene or delete DNA sequences at the site of DSB (Cheng et al., Genes Cells. 17:431-8, 2012). TALENs can be designed that target any safe harbor locus, such as AAVS1, CYBL, CCRS, and beta-globin.

In some embodiments, the nucleic acid encoding BCL11B operably linked to a promoter is delivered to the cell by a non-viral vectors (such as a plasmid vector). Electroporation can be used to introduce non-viral vectors into cells in vitro and in vivo. Generally, in this method, a high concentration of vector DNA is added to a suspension of host cell and the mixture is subjected to an electrical field of approximately 200 to 600 V/cm. Following electroporation, transformed cells are identified by any suitable means, such as growth on appropriate medium containing a selective agent. Electroporation has also been effectively used in animals or humans (see Lohr et al., Cancer Res. 61:3281-3284, 2001; Nakano et al, Hum Gene Ther. 12:1289-1297, 2001; Kim et al., Gene Ther. 10:1216-1224, 2003; Dean et al. Gene Ther. 10:1608-1615, 2003; and Young et al., Gene Ther. 10:1465-1470, 2003).

Another targeted delivery system for a polynucleotide encoding BCL11B is a colloidal dispersion system. Colloidal dispersion systems include macromolecule complexes, nanocapsules, microspheres, beads, and lipid-based systems including oil-in-water emulsions, micelles, mixed micelles, and liposomes. One colloidal dispersion system is a liposome. Liposomes are artificial membrane vesicles which are useful as delivery vehicles in vitro and in vivo. It has been shown that large unilamellar vesicles (LUV), which range in size from 0.2-4.0 microns, can encapsulate a substantial percentage of an aqueous buffer containing large macromolecules. RNA, DNA and intact virions can be encapsulated within the aqueous interior and be delivered to cells in a biologically active form (Fraley et al., Trends Biochem. Sci. 6:77, 1981). In order for a liposome to be an efficient gene transfer vehicle, the following characteristics should be present: (1) encapsulation of the nucleic acid of interest at high efficiency while not compromising their biological activity; (2) preferential and substantial binding to a target cell in comparison to non-target cells; (3) delivery of the aqueous contents of the vesicle to the target cell cytoplasm at high efficiency; and (4) accurate and effective expression of genetic information (Mannino et al., Biotechniques 6:682, 1988).

The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations.

Another targeting delivery system is the use of biodegradable and biocompatible polymer scaffolds (see Jang et al., Expert Rev. Medical Devices 1:127-138, 2004). These scaffolds usually contain a mixtures of one or more biodegradable polymers, for example and without limitation, saturated aliphatic polyesters, such as poly(lactic acid) (PLA), poly(glycolic acid), or poly(lactic-co-glycolide) (PLGA) copolymers, unsaturated linear polyesters, such as polypropylene fumarate (PPF), or microorganism produced aliphatic polyesters, such as polyhydroxyalkanoates (PHA), (see Rezwan et al., Biomaterials 27:3413-3431, 2006; Laurencin et al., Clin. Orthopaed. Rel. Res. 447:221-236). By varying the proportion of the various components, polymeric scaffolds of different mechanical properties are obtained. A commonly used scaffold contains a ratio of PLA to PGA is 75:25, but this ratio may change depending upon the specific application. Other commonly used scaffolds include surface bioeroding polymers, such as poly(anhydrides), such as trimellitylimidoglycine (TMA-gly) or pyromellitylimidoalanine (PMA-ala), or poly(phosphazenes), such as high molecular weight poly(organophasphazenes) (P[PHOS]), and bioactive ceramics. The gradual biodegradation of these scaffolds allows the gradual release of drugs or gene from the scaffold. Thus, an advantage of these polymeric carriers is that they represent not only a scaffold but also a drug or gene delivery system. This system is applicable to the delivery of plasmid DNA and also applicable to viral vectors, such as AAV or retroviral vectors, as well as transposon-based vectors.

In certain embodiments of the disclosed methods, the modified cells are incubated in vitro under conditions sufficient for differentiation and proliferation of T cells from the HSPCs and/or pluripotent stem cells, or proliferation of the mature T cells, prior to administering the cells to a subject. In any embodiment of the disclosed methods, modified cells may be administered to a subject at any time following the modification. In certain embodiments, the modified cells are incubated in vitro under such conditions for more than 14 days, or for more than 30 days, prior to administering the cells to a subject.

### IV. Methods of Use

Methods are provided herein for producing a T cell population for a T cell therapy, and also for treating a subject with a T cell therapy.

In some embodiments, a method is provided for producing a T cell population for a T cell therapy for a human subject. The method comprises providing HSPCs, pluripotent stem cells, or mature T cells as described herein, and increasing BCL11B expression in the HSPCs, pluripotent stem cells, or mature T cells as described herein to form modified cells with increased BCL11B expression compared to corresponding control cells. The increased BCL11B expression increases production and/or proliferation of T cells from the HSPCs or the pluripotent stem cells, or increases proliferation of the mature T cells, compared to the corresponding control cells, to form the T cell population for the T cell therapy. In several embodiments, the increase in production of T cells from the HSPCs or the pluripotent stem cells comprises an increase in the rate of production (for example, by at least 50%, such as at least 75%, at least 100%, at least 200%, at least 300%, at least 400%, or at least 500%) of the T cells in vitro or in vivo compared to control cells without the increase in BCL11B expression.

In further embodiments, the modified HSPCs, pluripotent stem cells, or mature T cells, or the T cells produced from proliferation of the modified HSPCs or the pluripotent stem cells, or the mature T cells are administered to the subject for the T cell therapy. Administration of modified cells with increased BCL11B expression to a subject can be accomplished by any suitable route, such as intravenous, intramuscular, intra-articular, and/or intrathecal (lumbar puncture) administration. Administration can be local or systemic. For example, if the chosen route is intravenous, the composition is administered by introducing the composition into a vein of the subject.

In some embodiments, the modified HSPCs, pluripotent stem cells, or mature T cells are prepared from cells obtained from the same subject to whom the cells are to be administered, and thus are autologous. The modified HSPCs, pluripotent stem cells, or mature T cells can also be prepared from cells from a different subject, and be allogeneic. Typically, donor(s) and recipient(s) are immunologically compatible. Thus the modified HSPCs, pluripotent stem cells, or mature T cells can be allogeneic.

A number of tissues can provide a source of HSPCs, pluripotent stem cells, or mature T cells for use in the methods described herein, and HSPCs, pluripotent stem cells, or mature T cells can be isolated from these tissues using any suitable procedure. In non-limiting examples, the HSPCs, pluripotent stem cells, or mature T cells are isolated from the umbilical cord blood, the bone marrow, and/or the peripheral blood.

In some embodiments, the HSPCs, pluripotent stem cells, or mature T cells are isolated from other cells using suitable sorting methods, such as fluorescence activated cell sorting (FACS) based on cell-surface markers specific to the HSPCs, pluripotent stem cells, or mature T cells. Analysis of HSPC, pluripotent stem cell, or mature T cell markers can be performed using any suitable methods (e.g., flow cytometric analysis, Western blot analysis, RT-PCR, in situ hybridization, immunoflourescence, immunohistochemistry, etc.). Further, analysis of production and/or proliferation of T cells from the HSPCs or pluripotent stem cells, or the proliferation of the mature T cells may be performed using any suitable method.

In any embodiment described herein using pluripotent stem cells, cells derived from the pluripotent stem cells, such as a mesodermal progenitor cell or any cell derived from a pluripotent stem cell that is capable of maturing to a T cell, can be used in place of the pluripotent stem cells.

Exemplary uses for the modified cells with increased BCL11B expression disclosed herein include, but are not limited to, enhancing thymic T cell reconstitution post HSCT; increasing ex vivo generation of T cell precursors, which can be co-transplanted with HSPCs to improve post HSCT thymic T cell reconstitution; enhancing the function and/or persistence of, and/or preventing the exhaustion of engineered T cells, such as CAR T cells and/or TCR T cells, such as for immunotherapy applications; generating T cells from pluripotent stem cells for the ex vivo production of allogenic T cell immunotherapies; enhancing the ex vivo expansion of engineered T cells during the production of CAR and/or TCR transformed cells, for example to enable generation of functional T cells for immunotherapy applications; manipulating the frequency of T cell subsets (CD4 or CD8) and/or memory cell subtypes in CAR and/or TCR transformed T cells, for example to maximize efficacy of engineered T cell immunotherapies; and/or generating (ex vivo and/or in vivo) T-regulatory cells, such as for treatment of graft versus host disease and/or autoimmune disorders.

In some embodiments, the T cell therapy comprises T cell reconstitution following HSCT, and the method comprises administering to the subject a therapeutically effective amount of the modified HSPCs, pluripotent stem cells, or mature T cells with increased BCL11B expression, and/or the T cells produced from proliferation of the modified HSPCs or the pluripotent stem cells, and/or the mature T cells. Any suitable dose of the cells can be administered to the subject that promotes T cell reconstitution in the subject following HSCT. In some embodiments, at least 10³/kg, such as at least 10⁴/kg or at least 10⁵/kg, of the modified cells are administered to the subject. In some embodiments, from 10⁴/kg to 10⁸/kg of the modified cells, such as from 10⁴/kg to 10⁷/kg, from 10⁴/kg to 10⁶/kg, or from 10⁵/kg to 10⁷/kg of the modified cells are administered to the subject, for example about 10⁴/kg, about 10⁵/kg, about 10⁶/kg, about 10⁷/kg, or about 10⁸/kg of the modified cells are administered to the subject. The method improves T cell reconstitution in the subject (for example, as measured by concentration of mature T cells in peripheral blood at a designated time post-HSCT), such as by at least 5%, at least 10%, at least 15%, at least 20%, at least 25%, at least 30%, at least 50%, at least 75%, at least 90%, or at least 95% as compared to a response in the absence of the therapy. T cell reconstitution in the subject may also be measured by time to achieve a certain concentration of mature T-cells or TREC (T-cell receptor excision circles, a marker of thymopoiesis) in peripheral blood, or time to achieve T-cell immune function (as measured by T-cell responses to Candida, tetanus, or viral antigens) (Brink M R M van den, Velardi E, Perales M-A. Immune reconstitution following stem cell transplantation. Hematology. 2015 Dec. 5; 2015(1):215-9). In some embodiments, T cell reconstitution in the subject is achieved within one year of administering the modified cells to the subject, such as within 9 months, within 6 months, or within 3 months.

In some embodiments, the T cell therapy comprises CAR T cell therapy for treatment of cancer, and the method comprises administering to the subject a therapeutically effective amount of the modified HSPCs, pluripotent stem cells, or mature T cells with increased BCL11B expression, or the T cells produced from proliferation of the modified HSPCs or the pluripotent stem cells, or the mature T cells. Any suitable dose of the cells can be administered to the subject that promotes the CAR T cell therapy for treatment of cancer in the subject. In some embodiments, at least 10³/kg, such as at least 10⁴/kg or at least 10⁵/kg, of the modified cells are administered to the subject. In some embodiments, from 10⁴/kg to 10⁷/kg of the modified cells, such as from 10⁴/kg to 10⁶/kg, or from 10⁵/kg to 10⁷/kg of the modified cells are administered to the subject, for example about 10⁴/kg, about 10⁵/kg, about 10⁶/kg, or about 10⁷/kg of the modified cells are administered to the subject. In this embodiment, the cells are further modified to express the CAR. The T cells exhibit reduced exhaustion in the subject (for example, as determined by number of circulating T cells expressing the CAR at a designated time point post-administration, or by an assay specific to the type of the CAR, such as duration of B cell aplasia for CAR T-cells directed against B-cell antigens, Maude S L et al. N Engl J Med. 2018 01; 378(5):439-48), such as by at least 5%, at least 10%, at least 15%, at least 20%, at least 25%, at least 30%, at least 50%, at least 75%, at least 90%, or at least 95% as compared to corresponding control cells that lack the increase in BCL11B expression.

In some embodiments, the T cell therapy comprises TCR T cell therapy for treatment of cancer, and the method comprises administering to the subject a therapeutically effective amount of the modified HSPCs, pluripotent stem cells, or mature T cells with increased BCL11B expression, or the T cells produced from proliferation of the modified HSPCs or the pluripotent stem cells, or the mature T cells. Any suitable dose of the cells can be administered to the subject that promotes the TCR T cell therapy for treatment of cancer in the subject. In some embodiments, at least 10³/kg, such as at least 10⁴/kg or at least 10⁵/kg, of the modified cells are administered to the subject. In some embodiments, from 10⁴/kg to 10⁸/kg of the modified cells, such as from 10⁴/kg to 10⁷/kg, from 10⁴/kg to 10⁶/kg, or from 10⁵/kg to 10⁷/kg of the modified cells are administered to the subject, for example about 10⁴/kg, about 10⁵/kg, about 10⁶/kg, about 10⁷/kg, or about 10⁸/kg of the modified cells are administered to the subject. In this embodiment, the cells are further modified to express the TCR. The T cells exhibit reduced exhaustion in the subject (for example, as determined by number of circulating T cells expressing the TCR at a designated time point post-administration), such as by at least 5%, at least 10%, at least 15%, at least 20%, at least 25%, at least 30%, at least 50%, at least 75%, at least 90%, or at least 95% as compared to corresponding control cells that lack the increase in BCL11B expression.

In some examples, the modified cells are administered to a subject that is human subject with an autoimmune disorder, such as, for example, rheumatoid arthritis is an autoimmune disorder, as are Hashimoto's thyroiditis, pernicious anemia, Addison's disease, type I diabetes, systemic lupus erythematosus, dermatomyositis, Sjogren's syndrome, dermatomyositis, lupus erythematosus, multiple sclerosis, myasthenia gravis, Reiter's syndrome, graft-vs-host disease, and/or Grave's disease.

Modified cells disclosed herein can be administered to a subject in combination with one or more additional therapeutics, such as, for example, one or more anti-cancer agents, antibiotics, and/or immunotherapeutics for treating cancer, infection, or autoimmune diseases.

### EXAMPLE

The following examples are provided to illustrate particular features of certain embodiments, but the scope of the claims should not be limited to those features exemplified.

### Example 1

BCL11B overexpression induces T-cell differentiation of multilineage human hematopoietic stem and progenitor cells

This example illustrates the effects of overexpressing the transcription factor BCL11B in human HSPCs and T cells in vitro.

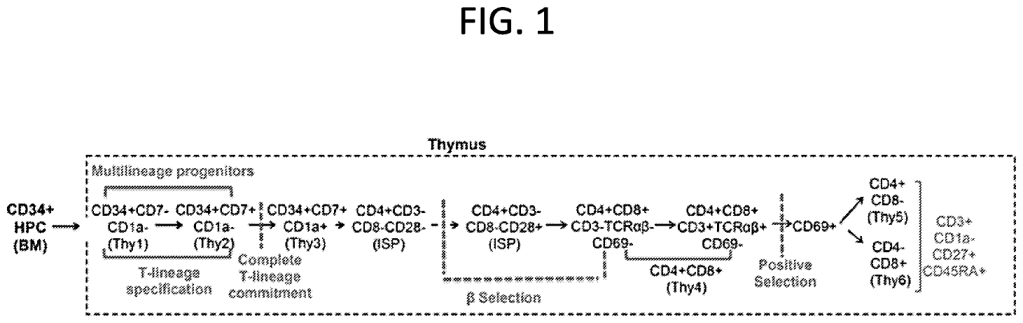

HSPCs that migrate from the bone marrow (BM) and initiate T cell differentiation in the human thymus (thymopoiesis) can be characterized by expression of the CD34 antigen and may comprise less than 1% of all thymocytes. The initial stages of thymopoiesis are marked by two processes: the induction of expression of T-lineage genes (T-lineage specification), and the loss of alternative (non-T) lineage potentials (T-lineage commitment). The earliest thymic progenitors (CD34+CD7−CD1a−, Thy1; FIG. 1) possess myelo-erythroid as well as full lymphoid (B, T, and NK) potential. Successive stages of T-lineage commitment are marked by sequential upregulation of CD7 and CD1a and progressive loss of alternative lineage potentials, resulting in the generation of CD34+CD7+CD1a+ cells (Thy3). These resulting cells are the earliest known fully T-lineage committed progenitors and can subsequently give rise to immature single positive (ISP, CD3−CD4+CD8−) cells. Further, CD34+CD7−CD1a− and CD34+CD7+CD1a− cells express T-lineage genes, indicating that specification may occur prior to complete commitment. ISP cells expressing a rearranged TCR (T cell receptor) 13 chain proliferate through pre-TCR signaling and differentiate into double positive (DP, CD4+CD8+) cells ((3-selection). Only those DP cells expressing a TCRαβ receptor reactive to a “self” peptide/MHC (major histocompatibility antigen) complex survive (positive selection) and differentiate into mature single positive CD3+ T cells (CD4+ and CD8+). Cells with high TCR reactivity to self peptides are eliminated via negative selection.

While many features of thymopoiesis are conserved between humans and mice, several species related differences exist in regulatory mechanisms underlying thymopoiesis. Therefore, the studies disclosed in this Example investigated human thymopoiesis and T cells to identify clinically relevant approaches for the improvement of human T cell differentiation and function. NOTCH1 signaling is required for murine T-lineage commitment and subsequent differentiation from the DN3 to DN4 stage during (3-selection. In contrast, a reduction in NOTCH signaling is likely required for human T-lineage commitment, and NOTCH1 signaling is likely required for proliferation, but not differentiation, during human (3-selection. Species related differences are also seen in the effects of overexpression of the T cell transcription factors TCF7, GATA3, and NOTCH1 in the context of differentiation of multilineage hematopoietic progenitors. Unlike in mice where Tcf7 is likely sufficient for induction of T-lineage genes even in the absence of NOTCH1 signals, TCF7 expression in the absence of NOTCH1 signals does not induce a T-lineage transcriptional program in human multilineage progenitors. Gata3 overexpression induces cell death in murine thymic progenitors, while it promotes commitment and differentiation into DP cells in human thymic progenitors. Sustained NOTCH1 signaling induces generation of TCRαβ T− cells in mice but leads to diversion of cells into the γδ rather than αβ T-lineage in human progenitors. An incomplete understanding of the mechanisms underlying human T cell differentiation and of species-related differences in the effects of overexpression of transcription factors on T-lineage differentiation have hindered development of methods to enhance human T-lineage differentiation and function.

Bcl11b is a transcription factor whose expression during murine hematopoiesis is restricted to the T and innate lymphoid lineages. Bcl11b knockout murine progenitors can upregulate T-lineage genes but fail to repress stem cell, natural killer cell (NK), and myeloid genes, and show a pre-commitment differentiation arrest. Bcl11b deletion after T-lineage commitment impairs positive selection and T cell function. In humans, BCL11B is not expressed in bone marrow HSPCs. BCL11B expression is first induced in the earliest CD34+ progenitors (CD34+CD7−CD1a−) in the thymus and is then upregulated with successive stages of T-lineage commitment and further differentiation into DP cells. BCL11B plays an important role in human T-lineage commitment and BCL11B regulatory activities differ between the initial stages of human and murine thymopoiesis. In contrast to murine Bcl11b knockout (KO) progenitors, human BCL11B knockdown (KD) T cell precursors not only failed to repress stem cell, NK, and myeloid genes, but also downregulated T-lineage genes. However, that a given transcription factor is required for T cell differentiation does not necessarily mean that overexpression of the factor will enhance T cell differentiation. For example, NOTCH1 is required for T cell differentiation but NOTCH1 overexpression inhibits TCR αβ+ T cell generation. Similarly, GATA3 is required for T-lineage differentiation but GATA3 overexpression results in reduced thymic cellularity.

The present studies show for the first time that BCL11B overexpression enhances or accelerates differentiation of human hematopoietic progenitor cells into mature T cells and improves the function of primary T cells. BCL11B overexpression accelerated T-cell differentiation of human HSPC including the expedited and enhanced generation of mature T-cells. Early transcriptional effects of BCL11B in multilineage HSPC included the induction of multiple T-cell genes and the repression of alternative (non-T) lineage TFs. Furthermore, overexpression was sufficient for the initiation of T-lineage differentiation from HSPC in the absence of NOTCH1 signaling. Mature naïve T-cells generated from BCL11B overexpressing HSPC showed enhanced proliferation and differentiation into cells with a central memory immunophenotype in response to CD3/CD28 activation. Our results reveal species-specific TF insights about the human T-cell differentiation that indicate BCL11B pathway activation as a potential strategy for enhancing post-HSCT T-cell reconstitution and improving the function of engineered T-cells in the context of immunotherapy approaches.

Exemplary uses for BCL11B overexpression in T cells can include enhancing thymic T cell reconstitution post HSCT; increasing ex vivo generation of T cell precursors, which can be co-transplanted with HSPCs to improve post HSCT thymic T cell reconstitution; enhancing the function and persistence and preventing the exhaustion of engineered T cells (CAR T cells and/or TCR T cells) for immunotherapy applications; generating functional T cells from pluripotent stem cells for the ex vivo production of allogenic T cell immunotherapies; enhancing the ex vivo expansion of engineered T cells during the production of CAR and TCR transduced cells to enable generation of functional T cells for immunotherapy applications; manipulating the frequency of T cell subsets (CD4 or CD8) and/or memory cell subtypes in CAR or TCR transduced T cells to maximize efficacy of engineered T cell immunotherapies; and/or generating (ex vivo and/or in vivo) T-regulatory cells for treatment of graft versus host disease and/or autoimmune disorders.

### Results

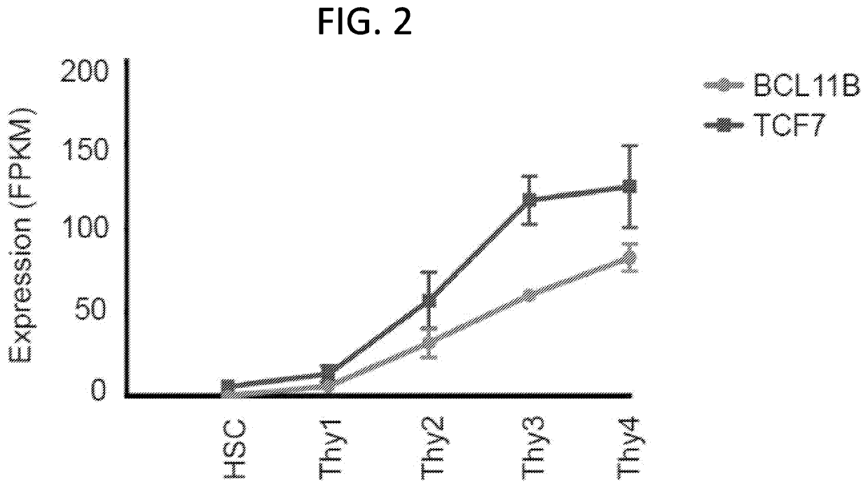

Species related differences exist in the expression profiles of BCL11B and TCF7 during thymopoiesis between humans and mice. In mice, Bcl11b expression is induced during the DN2a stage, by which point T-lineage specification and expression of Tcf7 and Gata3 have already occurred; subsequent Bcl11b expression upregulation is accompanied by little change in Tcf7 expression (Kueh et al., Nat Immunol. 2016; 17(8):956-65). The earlier onset of Tcf7 upregulation relative to Bcl11b is consistent with a role for Tcf7, but not Bcl11b, in T-lineage specification in mice. To assess the expression profiles of these transcription factors relative to each other during human thymopoiesis, the present study analyzed published RNA-Seq data from bone marrow-derived hematopoietic stem cells (HSC) as well as CD34+ progenitors and the more differentiated CD4+CD8+ cells (double positive, DP) from the human thymus (Casero et al., Nat Immunol. 2015 December; 16(12):1282-91). In contrast to findings reported in mice, in humans, BCL11B expression was first seen in the earliest thymic progenitors, and subsequent BCL11B upregulation was accompanied by concomitant upregulation of TCF7 (FIG. 2). Differences in the relative expression kinetics of these T-lineage transcription factors between humans and mice suggest species related differences in the effects of these transcription factors in the context of T-lineage specification during the initial stages of thymopoiesis.

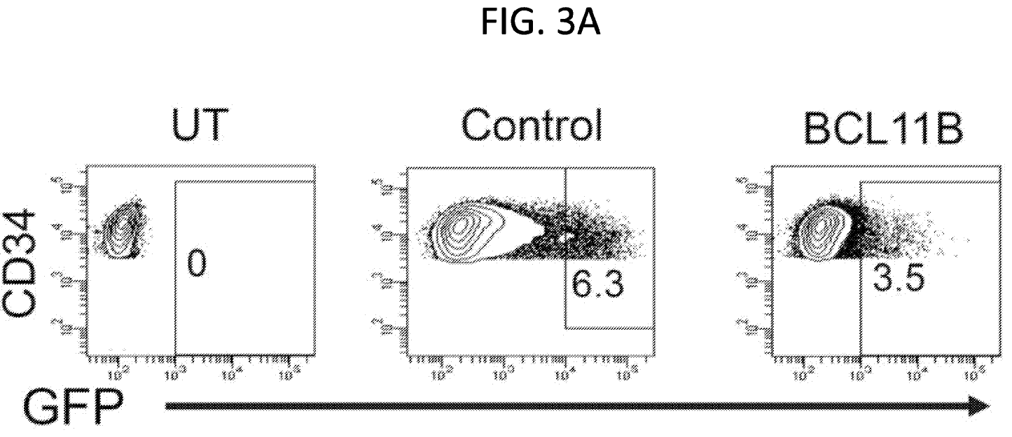

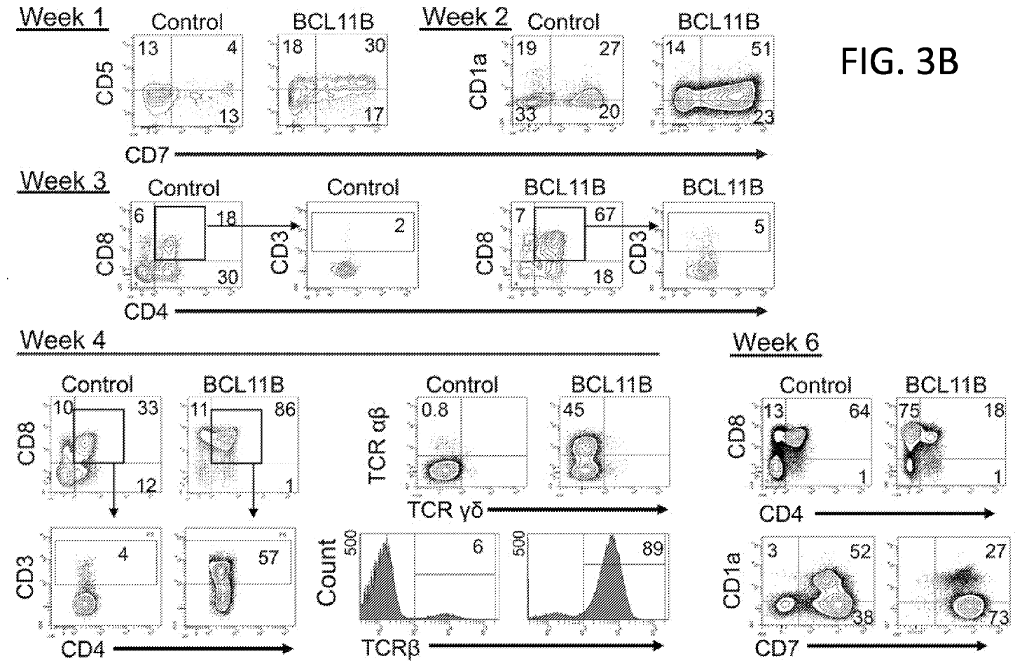

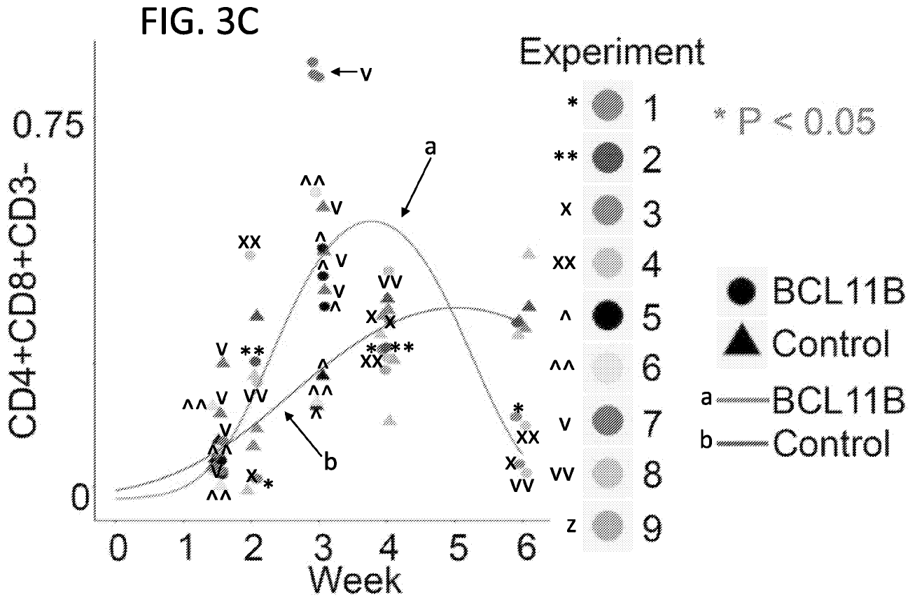

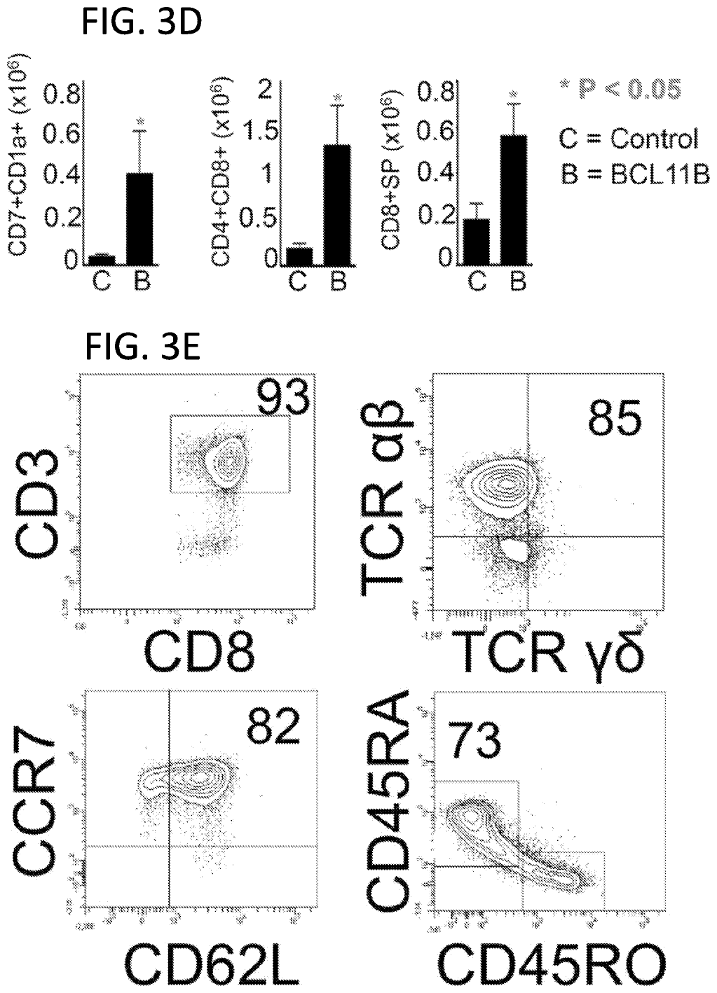

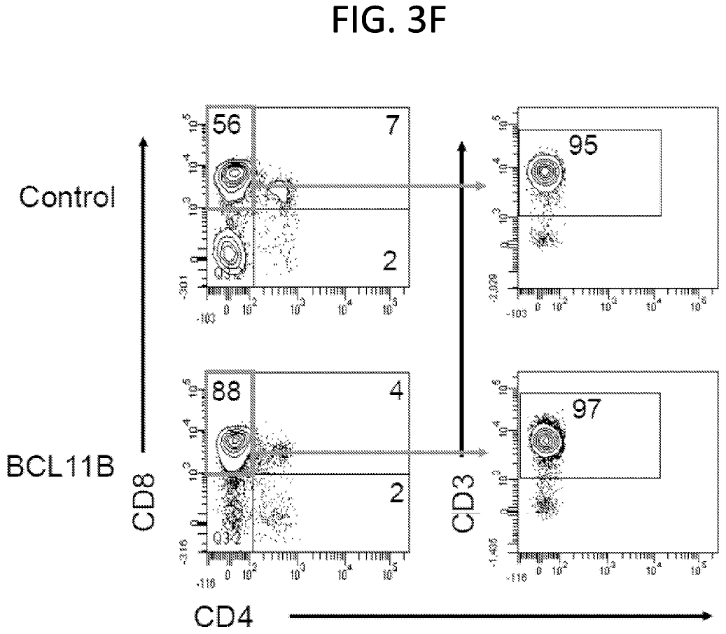

BCL11B gain of function enhances T-lineage differentiation of human HSPC. To determine whether BCL11B gain of function enhances T-lineage differentiation of human HSPC, overexpression experiments were performed in multilineage CD34+ cord blood (CB) HSPC using an in vitro three-dimensional artificial thymic organoid (ATO) co-culture model. ATOs, which comprise the MS5 stromal cell line transduced to express the NOTCH1 ligand DLL1 (MS5-DLL1), efficiently recapitulate the serial stages of thymopoiesis from human CD34+ HSPC (Seet et al, Nat Methods. 2017 May; 14(5):521-30). CB CD34+ cells were transduced with control (GFP) or BCL11B lentivirus. A low multiplicity of infection (=1) was used for lentiviral transduction, which results in BCL11B expression levels in cells transduced with BCL11B lentivirus that range from those seen in CD34+ primary human thymus cells to approximately three times that in CD4+CD8+ human thymus cells. Transduced (sorted CD34+Lin-GFP+) cells were cultured in ATOs (BCL11B or control ATOs) (FIG. 3A).

In control ATOs, CD7+ cells were seen on day 7 but only minimal differentiation into early T-cell precursors (CD5+CD7+) was observed at this early time point. Cells co-expressing CD7 and CD1a (CD7+CD1a+), an immunophenotype associated with T-lineage commitment, appeared in control ATOs by day 10 (approximately 15% of cells) and accounted for approximately 35% of cells on day 14. Furthermore, CD4+CD8−CD3− cells (immature single positive, ISP) accounted for approximately 25% of the cells in control ATOs on day 14. On day 21, control ATOs showed increased CD4+CD8+ cells (double positive, DP, approximately 20% of cells) and these cells did not express CD3 (CD3− DP). DP cells serially increased over time (approximately 40% of cells at day 28) to become the dominant population in control ATOs at day 42 (more than 50% of cells). The more differentiated CD3+TCRαβ+ DP cells, which first emerged on day 28, constituted a third or more of the cells in control ATOs on day 42. Consistent with the known bias toward CD8 single positive (SP) differentiation in the ATO system, CD3+TCRαβ+ SP cells were largely made up of CD8+ cells. CD8+ SP, first seen in on day 42 (approximately 15% of cells), increased over time to represent greater than 50% of the cells in control ATOs by day 84 (FIGS. 3B, 3F).

The frequencies of the different cell types observed in control HSPC ATOs at each time point were consistent with the published differentiation kinetics of CB CD34+ cells in ATOs. In contrast, ATOs initiated with BCL11B HSPC showed strikingly faster T-cell differentiation including the accelerated generation of CD8+ SP cells (% CD8+ SP in BCL11B vs. control ATOs=50% vs 10% at day 42 and 80% vs. 50% at day 84, FIGS. 3B, 3F). BCL11B induced the earliest stage of T-cell differentiation from HSPC as shown by a higher frequency of CD5+CD7+ cells in BCL11B ATOs relative to control ATOs on day 7. Furthermore, unlike in control ATOs, CD7+CD1a+ cells formed the predominant population in BCL11B ATOs by day 14 (approximately 50% of cells). On day 21, BCL11B ATOs were largely made up of DP cells, a cell type distribution not seen in control ATOs until day 42. CD3+TCRαβ+ cells represented almost half of the cells in BCL11B ATOs as early as day 28, a cell fraction substantially higher than that seen in control ATOs at the same timepoint. Overall, the differentiation time course curve was shifted to the left by approximately 1 week in BCL11B ATOs relative to control ATOs (p<0.05 for kinetics of differentiation of BCL11B vs. Control) (FIGS. 3B-3C).

BCL11B overexpression also significantly increased the output of SP T-cells. Outputs of cell types at preceding stages of T-lineage differentiation were also higher in BCL11B ATOs relative to control ATOs at the same time-points (FIG. 3D). SP cells arising from BCL11B HSPC showed a naïve mature T-cell phenotype (CD45RA+CCR7+CD62L+CD1a−) similar to that of SP cells generated by control HSPC (FIG. 3E). Overall, BCL11B gain of function enhanced and expedited T-cell differentiation of human HSPC.

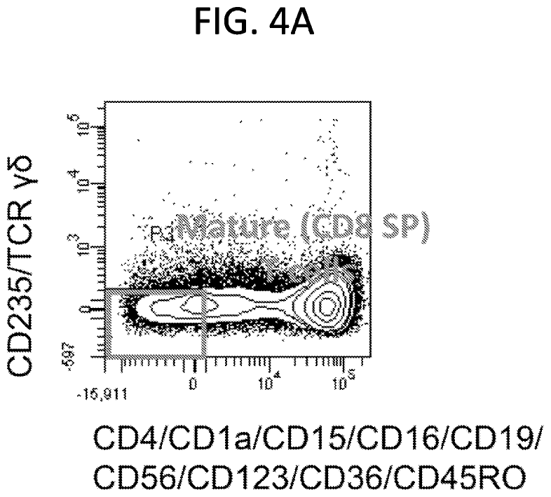

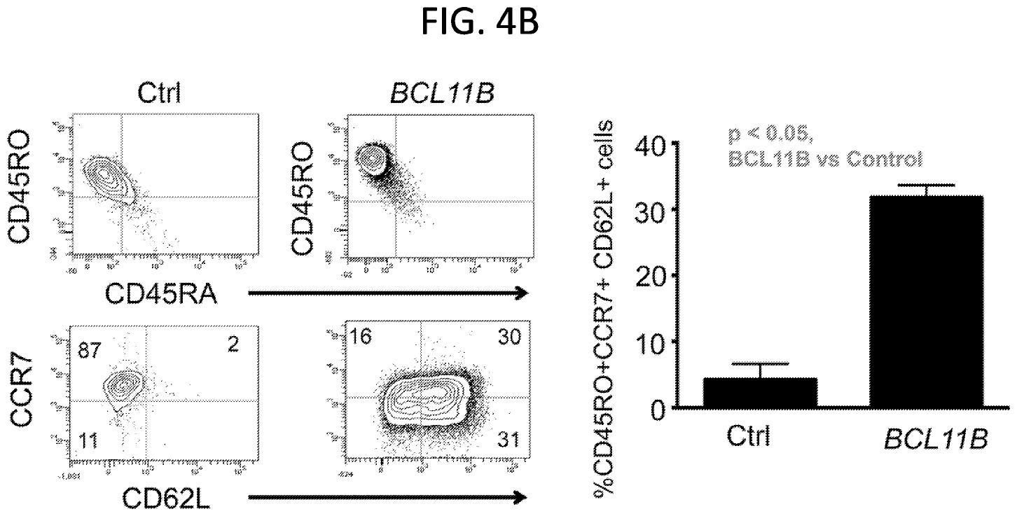

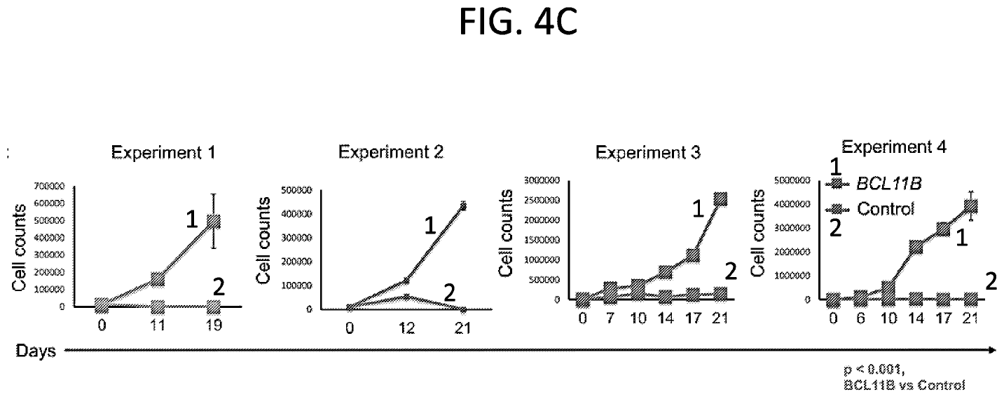

T-cells derived from BCL11B overexpressing HSPC show enhanced proliferation and differentiation into cells with a central memory immunophenotype. To investigate functional responses of the BCL11B-overexpressing, HSPC-derived T-cells to T-cell receptor (TCR) pathway activation, naïve (CD45RO−) SP T-cells from BCL11B or control ATOs were isolated using FACS (FIG. 4A). Sorted T-cells were stimulated with anti-CD3/CD28 beads and IL-2.

Both control and BCL11B T-cells upregulated the T-cell activation marker CD45RO. However, control T-cells showed minimal differentiation into cells with a central memory immunophenotype (CCR7+CD62L+) (Mahnke et al., Eur J Immunol. 2013 November; 43(11):2797-809). In contrast, BCL11B T-cells showed significantly higher cell output and robust differentiation into cells with a central memory immunophenotype (mean CCR7+CD62L+CD45RO+ cells=30% vs. 4% for BCL11B vs. control, p<0.05) (FIGS. 4B-4C). Furthermore, consistent with the higher frequency of central memory immunophenotype cells, activated BCL11B T-cells showed more sustained proliferation relative to control T-cells in response to repeated stimulation with anti-CD3/CD28 beads (FIG. 4C). Overall, these results are consistent with enhanced TCR functional responses in BCL11B-overexpressing, HSPC-derived T-cells.

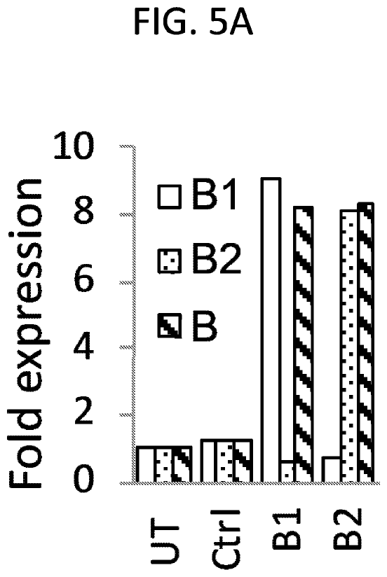

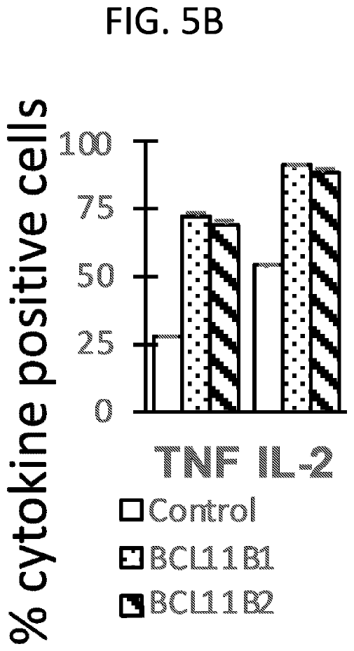

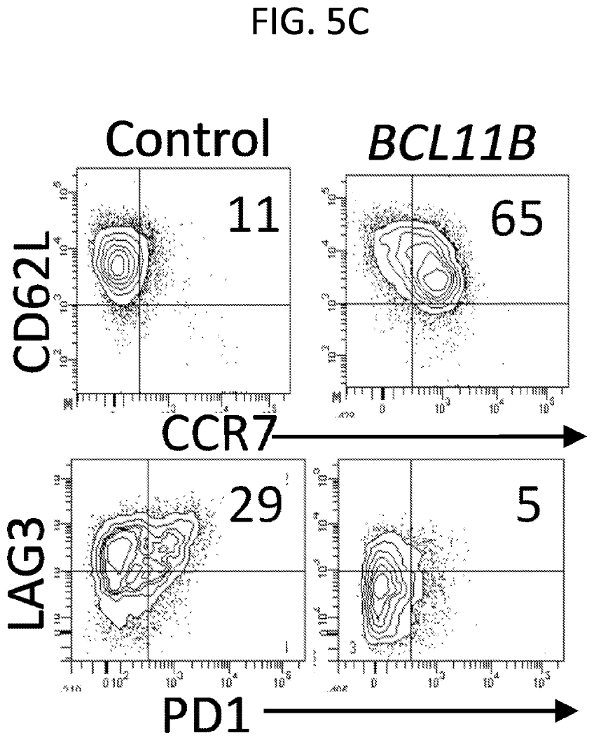

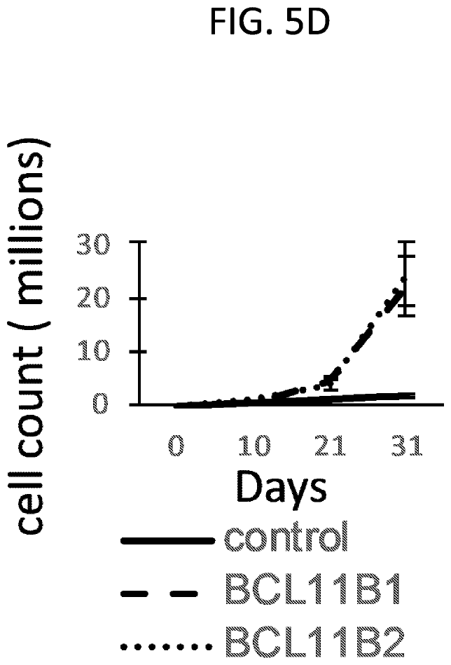

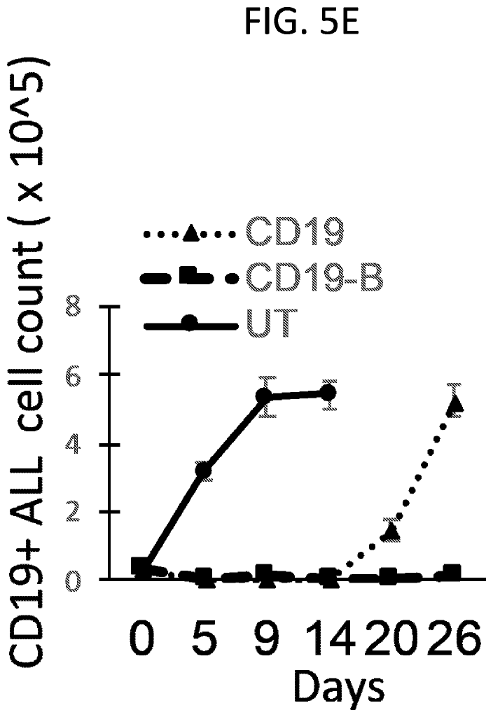

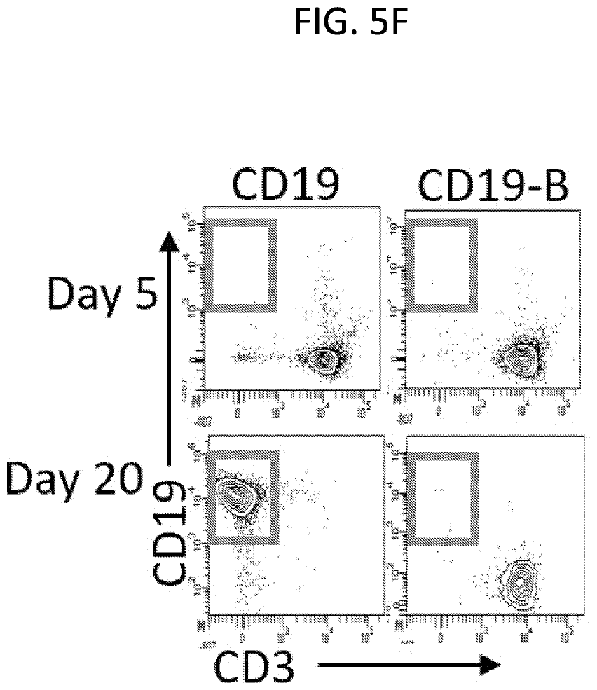

BCL11B overexpression in mature T cells enhances the functional responses of T cells to stimulation and mitigates their exhaustion in response to repeated activation. Given the enhanced function of the T cells generated when BCL11B is overexpressed at all stages of thymopoiesis, including the initial HSPC stage, this study investigated whether overexpressing BCL11B in differentiated, mature T cells would enhance T cell function. T cells were isolated from human peripheral blood and transduced with BCL11B or control lentivirus. Transduced cells were stimulated with PMA/ionomycin and CD3/CD28 to determine cytokine and proliferative responses to activation, respectively. BCL11B T cells showed higher production of TNFα and IL-2 than control T cells. BCL11B T cells repeatedly stimulated via activation of TCR pathway signaling showed greater and more sustained expansion, lower expression of T cell exhaustion markers, and higher frequencies of cells with a central memory immunophenotype as compared to control T cells (FIG. 5). T cells transduced with BCL11B vector at a multiplicity of infection of 1 or 5 showed robust proliferation, while cells transduced with MOI=10 failed to proliferate, indicating that the effects of BCL11B on T cell proliferation are BCL11B expression level specific.

T cells co-transduced with an anti-CD19 CAR and BCL11B showed a more sustained ability to eliminate B-ALL cells than control CD19 CAR T cells when repeatedly stimulated with ALL cells (FIG. 5). In summary, BCL11B overexpression enhanced the functional responses of T cells to stimulation and mitigated their exhaustion in response to repeated activation.

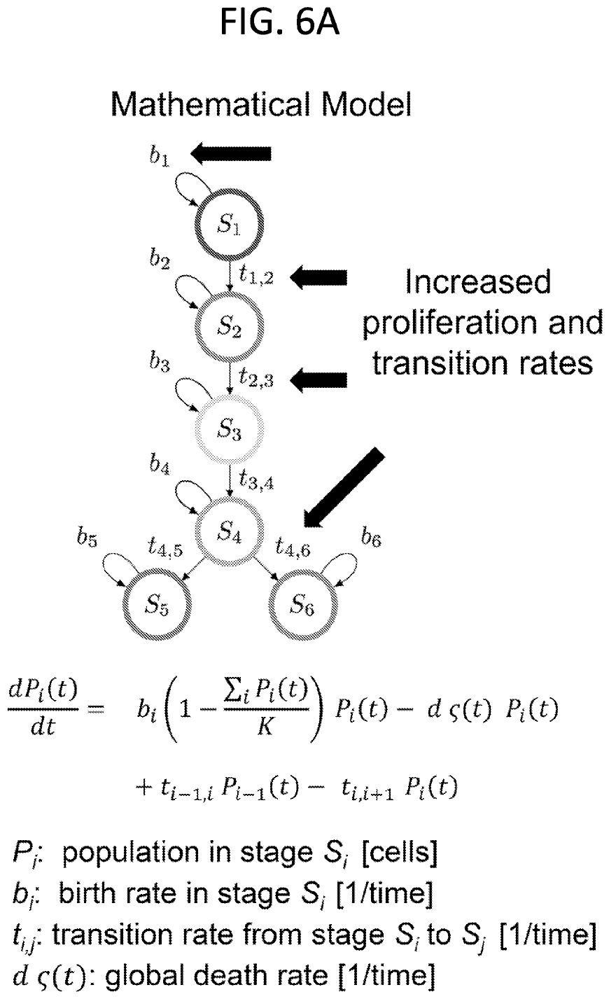

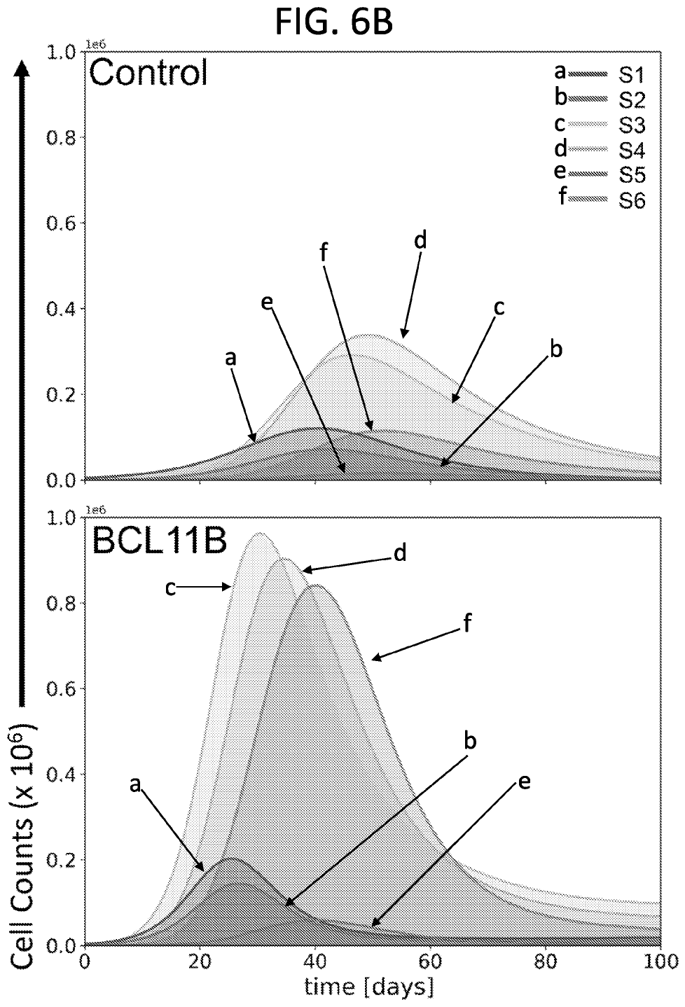

BCL11B-overexpressing cells show accelerated differentiation at multiple cell state transitions during T-cell differentiation. With respect to the frequency and number of cells at a given stage of differentiation, potential factors driving the observed differences between control and BCL11B ATOs include the effects of BCL11B on proliferation, survival, and/or cell state transitions, and/or the effects of BCL11B on the generation of cells at preceding stages. To decipher these factors and identify which stages of T-cell differentiation are enhanced by BCL11B, the dynamics of differentiation of control and BCL11B cells were mathematically modeled. The mathematical model employed ordinary differential equations that predict the number of cells at each of 6 stages of T-cell differentiation (CD4−CD8− [DN], ISP, CD3− DP, CD3+DP, CD4SP, and CD8SP) as a function of time that includes proliferation rate, transition rate, and death rate parameters (FIG. 6).

Model parameters were first estimated from control ATO experimental data. In the thymus, SP T-cells tend to be non-proliferative. Proliferation rates are highest in DP cells undergoing β-selection (CD3− DP) and are low in cells undergoing positive selection (CD3+DP). Constraints that mirror the relations between these proliferation rates in normal thymopoiesis (b₃>b₂≈0.8 b₃>b₁≈0.5 b₃>b₄≈0.3 b₃; b₅=0; b₆=0; b₁₋₆: proliferation rates for DN, ISP, CD3-DP, CD3+DP, CD4SP, and CD8 SP, respectively) did not affect the model's ability to fit the observed differentiation kinetics seen in control ATOs. However, these proliferation constraints were incompatible with the experimental data from BCL11B ATOs, indicating the model's ability to capture the differences in differentiation dynamics between BCL11B and control ATOs.

Next, effects of BCL11B were inferred by altering parameters for each stage to fit model predictions to the data from BCL11B cells. Modeling results predicted that effects of BCL11B at the DN stage alone were not sufficient to explain the observed differences between control and BCL11B cells (FIG. 6). Further, BCL11B most likely also enhanced the differentiation of ISP cells into CD3− DP cells and that of CD3+ DP cells into SP CD8+ cells (FIG. 6). Overall, these results suggest that BCL11B not only induces T-lineage differentiation of multilineage HSPC but also expedites the post-commitment stages of T-cell differentiation.

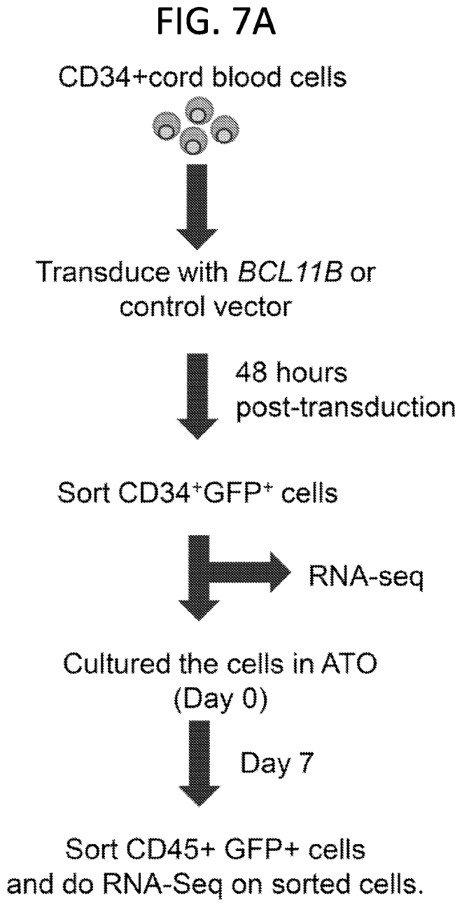

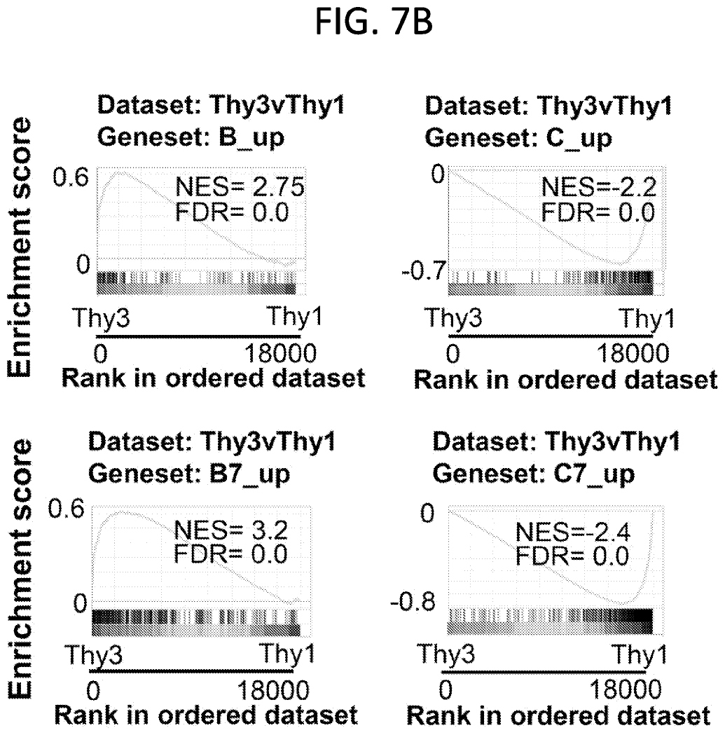

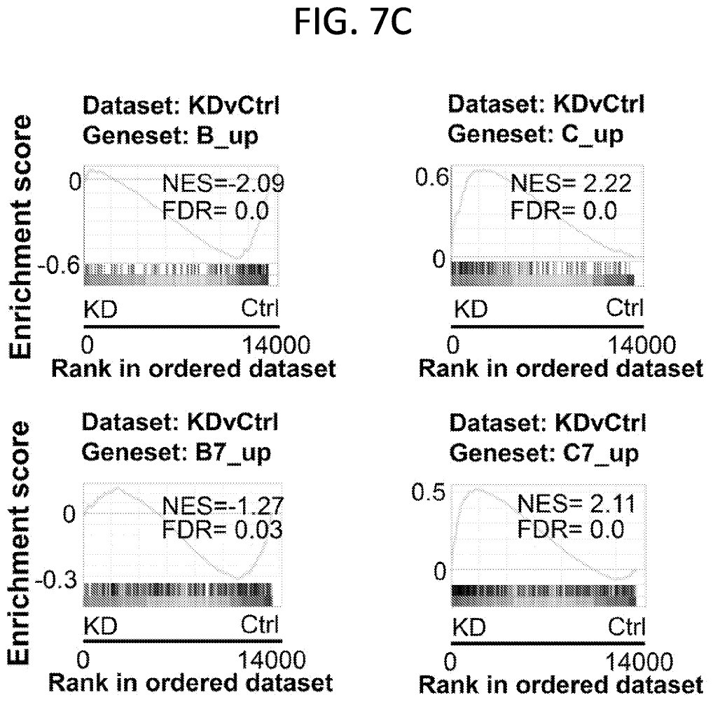

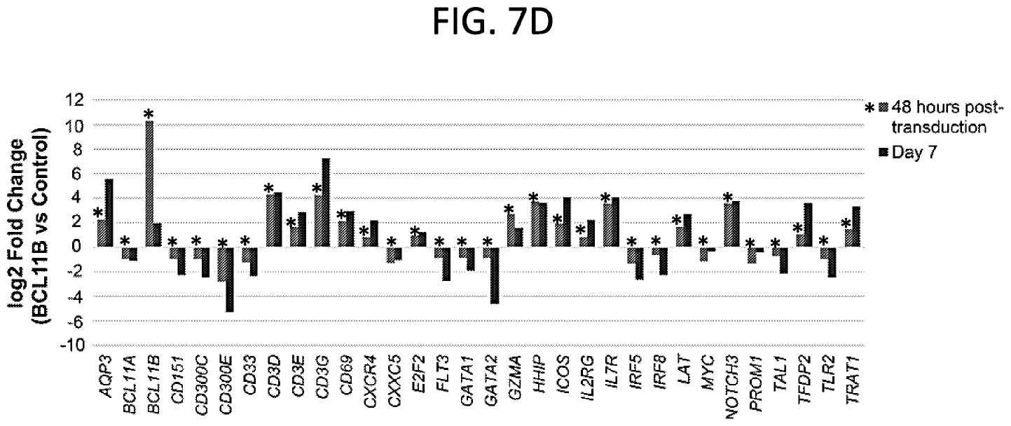

BCL11B overexpression in HSPC acutely induces a T-cell transcriptional program and represses alternative lineage programs. To determine potential direct transcriptional effects of BCL11B in HSPC, the present study investigated gene expression changes in HSPC after transduction with BCL11B lentivirus. Whole transcriptome profiling (RNA-Seq) of RNA extracted from CD34+GFP+lin− cells sorted 48 hours post-transduction of HSPC with BCL11B or control lentivirus was performed. HSPC were cultured on retronectin (stroma free culture without NOTCH1 ligand) during these 48 hours. Cells that were only briefly cultured (48 hours) and not exposed to stroma were used to minimize indirect transcriptional effects secondary to differentiation and thereby determine the acute effects of BCL11B on gene expression in the absence of NOTCH1 signaling. In addition, RNA-Seq was performed using CD45+GFP+ cells sorted from ATOs initiated with BCL11B or control HSPC (cells sorted 7 days after creating the ATOs). Cells sorted from ATOs were used to determine the effects of BCL11B on gene expression in the presence of NOTCH1 signaling (FIG. 7A).

BCL11B induced the upregulation of multiple genes associated with T-cell differentiation, including NOTCH3, IL7R, and IL2RG. Genes known to be upregulated with T-cell differentiation (CD3 genes, TRAT1, AQP3, CD69, and LCOS) showed increased expression in BCL11B cells relative to control cells. Furthermore, HSPC genes (BCL11A, TAL1, PROM1, and FLT3) and myeloid associated genes such as GATA1, GATA2, and IRF8 were repressed in BCL11B overexpressing HSPC (FIG. 7B-D). The transcriptional effects of BCL11B overexpression in HSPC showed substantial overlap with BCL11B dependent gene expression changes seen in previously reported BCL11B loss of function human T-cell differentiation studies. These transcriptional effects of BCL11B were seen as early as 48 hours and even in the absence of NOTCH1 signaling, and many of these effects were further enhanced in the presence of NOTCH1 signaling (day 7 ATOs) (FIG. 7B-D). Overall, these results support the notion that BCL11B initiates and establishes the T-lineage transcriptional program in human HSPC.

BCL11B is sufficient for the initiation of T-cell differentiation of human HSPC in the absence of NOTCH signaling. Since BCL11B overexpression accelerated the initial stages of T-cell differentiation from HSPC in ATOs (i.e. generation of CD5+CD7+ cells) and the DN to ISP transition, and BCL11B is required for T-lineage specification of human HSPC, this study investigated whether BCL11B is sufficient to induce T-cell differentiation of human HSPC in the absence of NOTCH1 signaling. CB HSPC transduced with BCL11B or control lentivirus were cultured in the presence (MS5-DLL1 ATO) or absence (organoids lacking delta-like ligands, i.e. made of MS5) of NOTCH1 signaling to determine if BCL11B could initiate T-cell differentiation.

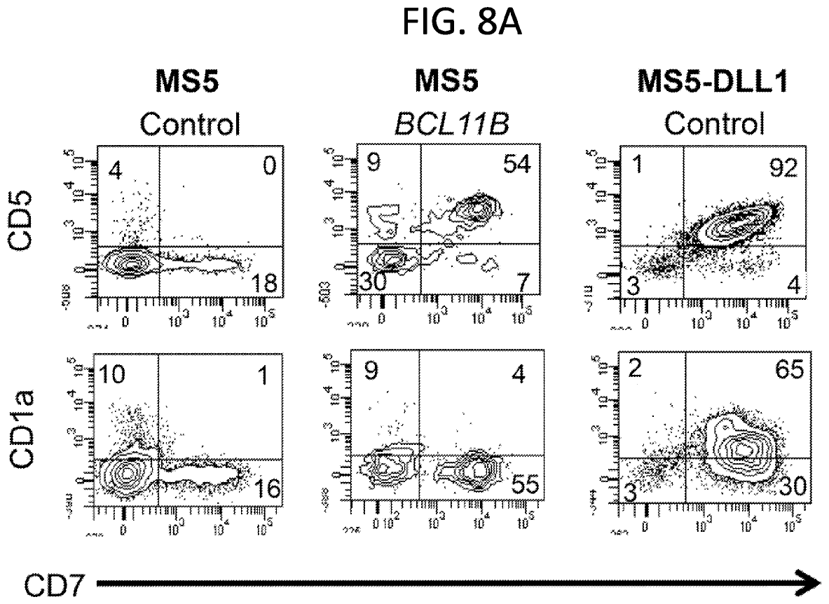

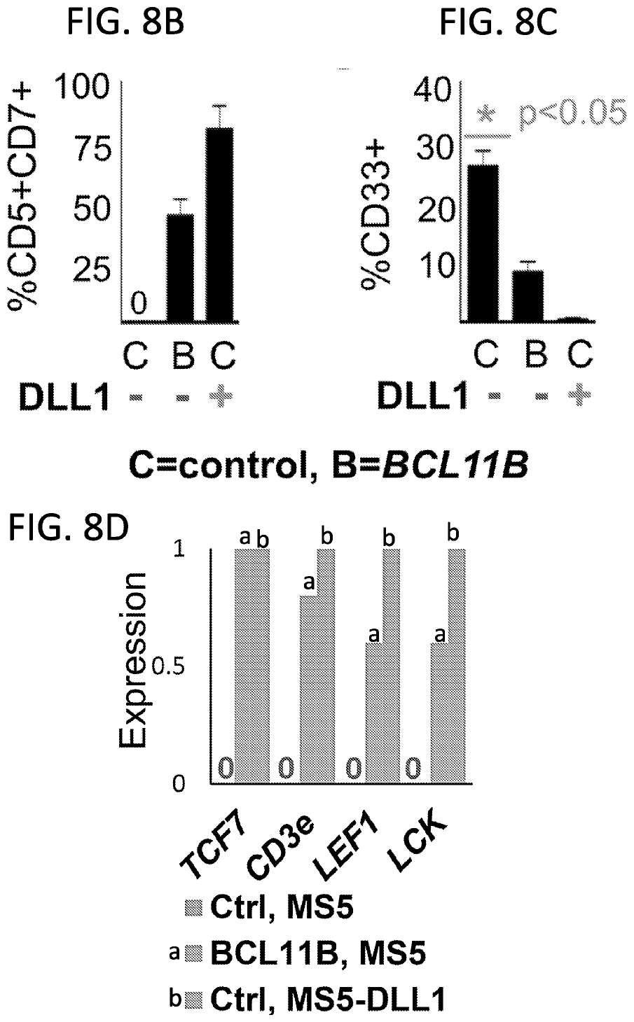

No T-cell precursors were generated in MS5 organoid cultures of control HSPC (FIG. 8A). In contrast, BCL11B HSPC generated early T-cell precursors (CD5+CD7+CD56−CD1a− cells) even in the absence of NOTCH1 signaling (FIGS. 8A-8B). BCL11B repressed myeloid differentiation in MS5 organoids (FIG. 8C). CD5+CD7+ cells generated in MS5 organoid cultures of BCL11B cells expressed the T-lineage genes TCF7, LCK, and LEF1 (FIG. 8D). However, BCL11B was not sufficient in the absence of NOTCH1 signaling for further differentiation into CD7+CD1a+ T-cell precursors (FIG. 8A). Overall, these data indicate that BCL11B is sufficient for the initiation of T-cell differentiation of human HSPC in the absence of NOTCH1 signaling but T-cell commitment requires additional regulatory inputs from NOTCH1

### DISCUSSION

Gain of function of Tcf7, Gata3, or Bcl11b, transcription factors important for the initial stages of thymopoieisis, have not been reported to enhance differentiation of murine HSPC into SP T-cells. While loss of function studies showed Bcl11b to be indispensable for repression of NK potential and thereby T-lineage commitment of murine HSPC, unlike Tcf7, Bcl11b is not a T-lineage specification transcription factor in mice (Li et al., Science. 2010 Jul. 2; 329(5987):89-93). Moreover, TCF7, GATA3, or NOTCH1 overexpression do not increase the generation of SP TCRαβ+ T-cells from human CB HSPC (Van de Walle et al., Nat Commun. 2016; 7:11171; De Smedt et al., J Immunology. 2002 Sep. 15; 169(6):3021-9). The enhanced differentiation of BCL11B-overexpressing HSPC into SP TCRαβ+ T-cells is thus a novel finding. Of note, BCL11B gain of function studies have not been possible in murine HSPC due to cell death of BCL11B-overexpressing cells. The findings disclosed herein are consistent with a species-specific role for BCL11B as a T-lineage specification transcription factor in humans with effects akin to that of Tcf7 in mice. These results emphasize the critical need for specifically studying human thymopoiesis to enable the translation of T-cell biology insights into therapeutic approaches for improving immune reconstitution in patients.

Previous knockdown studies suggested that BCL11B is required for T-lineage specification and commitment of human HSPC. However, results from loss of function studies are not necessarily predictive of the effects of overexpressing a given gene in the context of T-cell differentiation. For instance, while knockdown of GATA3 or inhibition of NOTCH1 signaling impairs or abrogates T-cell differentiation of human thymic progenitors respectively (Van de Walle et al., Nat Commun. 2016; 7:11171; Van de Walle et al., J Exp Med. 2013 Apr. 8; 210(4):683-97), gain of function of these genes inhibits the generation of TCRαβ+ cells (Van de Walle et al., J Exp Med. 2013 Apr. 8; 210(4):683-97; Taghon et al., J Immunol. 2001 Oct. 15; 167(8):4468-75). The need for precise, stage-specific regulation of the timing and level of expression of these genes during thymopoiesis may account for the paradoxical effects on T-cell differentiation when these genes are overexpressed.

The safety of lentivirus transduced HSPC in clinical trials and the advent of suicide switches to eliminate transduced cells support the feasibility of translating lentivirally modified HSPC to improve post HSCT immune reconstitution. Of note, overexpression of BCL11B, a tumor suppressor, inhibits the proliferation and induces apoptosis of T-ALL cells, data that support the safety, from an oncogenic perspective, of strategies involving BCL11B gain of function.

### Materials and Methods

Lentiviral vectors. The recombinant BCL11B expression lentiviral plasmid was generated by inserting a PCR amplified BCL11B cDNA sequence from the Open Reading Frame (ORF) of BCL11B plasmid (ThermoFisher Scientific, Waltham, Mass.) into the MNDU3-PGK-GFP expression vector using the In-Fusion® HD Cloning Kit (Clontech, Mountainview, Calif.). Plasmids were packaged into lentiviral particles by co-transfection with psPAX2 (Addgene, #12260) and pMD2.G (Addgene, #12259) plasmids into the 293FT using TranslT-293 transfection reagent (Mirus, MIR 2700). BCL11B expression and corresponding control (MNDU3-PGK-GFP) vectors were concentrated by ultracentrifugation (12000 rpm for 4 hours, at 4° C.).

Primary tissues. Deidentified Cord blood (CB) samples were obtained from University of California Los Angeles and Stemcyte (Pasadena, Calif., USA) and deidentified leucodepletion filters discarded after blood collection from donors were obtained from the Children's Hospital Los Angeles (CHLA) blood bank donor center in accordance with a CHLA Institutional review Board approved protocol. Peripheral blood T-cells were extracted from leucodepletion filters by washing out cells from the filter followed by ficoll separation of mononuclear cells and subsequent FACS (cells negative for CD1a, CD15, CD16, CD19, CD56, CD123, CD36, CD45RO, CD235, and TCR γδ) or magnetic activated cell sorting MACS, Miltenyi Biotec, San Diego, Calif.) enrichment for T-cells.

Transduction and culture of CB CD34+ cells and peripheral blood T-cells. CB CD34+ cells were enriched using magnetic activated cell sorting (MACS, Miltenyi Biotec, San Diego, Calif.). CD34+CB cells were cultured for 16 hours in 100 microliters of EX-Vivo 15 [Lonza, Walkersville, Md.] with thrombopoietin (50 ng/ml), FLT3 ligand (50 ng/ml], Stem cell factor [50 ng/ml], and 1-glutamine [2 mM, Cellgro, Manassas, Va.]) on retronectin (50 ng/ml, Clontech) coated non-tissue culture-treated 48-well plates (100,000 cells/well). Two doses of concentrated lentivirus (multiplicity of infection, MOI=1) were then added 24 hours apart. After 48 hours of exposure to lentivirus, CD34+GFP+CD3−CD4−CD8−CD56−CD19− (CD34+GFP+lin−) cells were sorted using fluorescence activation cell sorting (FACS) and then either analyzed by RNA-Seq or cultured in MS5-DLL1 (artificial thymic organoids, ATO) or MS5 organoids.

Peripheral blood T-cells were activated with CD3/CD28 beads (2 microliters per well) in Aim V medium (95% AIM V medium, 5% Human Serum AB, 25 ng/ml IL-2, 100,000 cells/well, 200 microliters of medium per well of a 96 well plate). Cells were transferred to retronectin (50 ng/ml, Clontech) coated non-tissue culture-treated 48-well plates (1:1 well to well transfer) at 24 hours post-activation. Concentrated lentivirus was added 6-24 hours after the transfer. A MOI of one (two doses 24 hours apart) or 5 (one dose) was used for single transduction experiments (BCL11B or control GFP vector). For the double transduction experiment, cells were co-transduced with BCL11B (single dose of MOI=S) and CD19 CAR lentivirus (two doses 24 hours apart of MOI=S) or singly transduced with CD19 CAR vector (control cells). Cells were cultured for a total of 7 days post-activation (cultures were split and re-plated with fresh AIM V medium upon confluency) and then sorted via FACS to isolate GFP+ live (DAPI−) cells for downstream experiments.

Organoid cultures. Sorted CB cells mixed with 150,000 MS5-DLL1 or MS5 cells were centrifuged, resuspended in 5-10 microliters of PBS+1% FBS, and deposited on a cell culture insert, which was then cultured in a 6-well plate containing T cell differentiation medium (94% RPMI, 4% B27 Supplement, 1% Glutamax, 1% Pen/Strep, 30 μm ascorbic acid, 5 ng/ml IL-7, 5 ng/ml FLT3-ligand) to create organoids (1 organoid per well, 1 ml of medium per well). Culture medium was replaced with fresh medium twice a week. Organoids were initiated with 2400-5000 sorted CB cells. In each experiment, equal numbers of CB cells were used to initiate organoids. Lineage differentiation in organoids was analyzed by flow cytometry. Following staining with surface antibodies, cells were fixed, permeabilized, and staining with TCRβ antibody for analysis of TCRβ expression. Table 1 lists FACS antibodies used.

TABLE 1
FACS Antibodies

| Experiment | Marker | Clone | Company | Catalog # |
|---|---|---|---|---|
| Flow Cytometry | CD1A APC-Cy7 | HI149 | Biolegend | 300125 |
| Analysis | | | | |
| | CD7 PE | CD7-6B7 | Biolegend | 343106 |
| | CD34 APC | 561 | Biolegend | 343607 |
| | CD45 PerCP | HI30 | Biolegend | 304026 |
| | CD3 APC | UCHT1 | Biolegend | 561810 |
| | CD4 PE-Cy7 | OKT4 | Biolegend | 317413 |
| | CD5 PE | UCHT2 | Biolegend | 300607 |
| | CD8 APC-Cy7 | SK1 | Biolegend | 344714 |
| | TCRαβ PE | IP26 | Biolegend | 306708 |
| | TCRγδ APC | B1 | Biolegend | 331211 |
| | CCR7 PE | 150503 | R&D | MAB197 |
| | CD62L APC | DREG-56 | Biolegend | 304809 |
| | CD45RA PE-Cy7 | HI100 | Biolegend | 304125 |
| | CD45RO PE | UCHL1 | Biolegend | 304205 |
| | CD3 PerCP | SK7 | Biolegend | 344813 |
| | CD8 APC-Cy7 | SK1 | Biolegend | 344714 |
| | CD45RA PE-Cy7 | HI100 | BD Bioscience | 560675 |
| | CD45RO PE | UCHL1 | BD Bioscience | 561889 |
| | CD62L APC | DREG-56 | Biolegend | 304809 |
| | CCR7 PE | 150503 | R&D | MAB197 |
| | CD3 AF 700 | UCHT-1 | BD Bioscience | 557943 |
| | CD8a BV421 | RPA-T8 | Biolegend | 301036 |
| | CD4 PE-Cy7 | RPA-T4 | Biolegend | 300511 |
| | CD5 APC | L17F12 | BD Bioscience | 340658 |
| | CD45 PerCP | 2D1 | Biolegend | 368505 |
| FACS Sorting for T | CD4 PE | A161A1 | Biolegend | 357409 |
| Cell Activation | | | | |
| | CD15 PE | H198 | Biolegend | 301905 |
| | CD16 PE | 3G8 | Biolegend | 302007 |
| | CD19 PE | 4G7 | Biolegend | 392505 |
| | CD36 PE | 5-271 | Biolegend | 336205 |
| | CD56 PE | HCD56 | Biolegend | 318306 |
| | CD123 PE | 6H6 | Biolegend | 306005 |
| | CD1a PE | H149 | Biolegend | 300106 |
| | CD45RO PE | UCHL1 | Biolegend | 304205 |
| | CD235a APC | GA-R2 (HIR2) | BD Bioscience | 561775 |
| | TCRγδ APC | B1 | Biolegend | 331211 |
| Sorting for | CD34 Pe-Cy7 | 581 | Biolegend | 343516 |
| CD34+/GFP+/lin− cells | | | | |
| | CD3 APC | UCHT1 | Biolegend | 300439 |
| | CD4 APC | RPA-T4 | BD Bioscience | 555349 |
| | CD8 APC | SK1 | Biolegend | 344722 |
| | CD19 APC | 4G7 | Biolegend | 392503 |
| | CD56 APC | NCAM16.2 | BD Bioscience | 341025 |

T-cell activation assay. Naïve mature GFP+CD8 SP T cells sorted from ATO at weeks 6-12 of culture or transduced (GFP+) human peripheral blood T-cells were activated with CD3/CD28 beads in Aim V medium (95% AIM V medium, 5% Human Serum AB, 20-25 ng/ml IL-2). CD8 SP T cells were isolated from ATO by a negative selection FACS approach (i.e. cells negative for CD4, CD1a, CD15, CD16, CD19, CD56, CD123, CD36, CD45RO, CD235, and TCR γδ). 10,000-20,000 sorted cells were activated in 200 microliters of medium per well of a 96 well plate. Cultures were split and re-plated with fresh AIM V medium upon confluency. The immunophenotype of activated cells in culture was analyzed by flow cytometry. CD3/CD28 beads were magnetically removed prior to staining with flow cytometry antibodies.

Mathematical modeling. A mathematical model was developed that describes the evolution of T-cells through the six differentiation stages in FIG. 6. This is expressed mathematically by a system of six coupled ordinary differential equations of the general form:

$$\frac{d {P}_{i} ( t )}{d t} = {b}_{i} ( 1 - \frac{{\sum }_{i} {P}_{i} ( t )}{K} ) {P}_{i} ( t ) - d ς ( t ) {P}_{i} ( t ) + {t}_{i - 1 , i} {P}_{i - 1} ( t ) - {t}_{i , i + 1} {P}_{i} ( t )$$

Each equation describes the temporal change of cells in one differentiation stage Pᵢ (t) [cells] in terms of proliferation and death of cells in that stage, as well as differentiation into and out of this stage. The parameters of the model correspond to (1) proliferation rates bᵢ [day⁻¹] of cells in each stage, (2) transition rates t_{i,j} [day⁻¹] between subsequent stages, and (3) a global death rate d [day⁻¹] for cells across stages.

Cell proliferation in each stage is modeled as a logistic growth process with carrying capacity K to account for constraints on overall population size and growth due to spatial limitations and finite NOTCH1 signaling in ATO. Based on the published knowledge about stage specific proliferation rates during thymopoiesis, this study assumed b₃>b₂≈0.8 b₃>b₁≈0.5 b₃>b₄≈0.3 b₃ for control cells. The observed rapid decrease of total cell population after about 6 weeks was modeled by imposing a time-dependency ç(t) on the death rate d. This time-dependency was modeled as an increasing sigmoidal function that reaches ç(t)=1/2 at a critical time t=40 days and approaches ç(t)=1 for large time t.

Starting from a model parameterization that is representative for the control group, we identified the minimum set of changes in model parameters that could reproduce typical characteristics of the BCL11B differentiation dynamics.

RNA-Seq. RNA-Seq was performed on FACS sorted CD34+GFP+lin− cells (sorted 48 hours post-transduction) or CD45+GFP+ cells (sorted from ATO 7 days after initiation of cultures). The Arcturus Picopure RNA extraction kit or the Qiagen MIrneasy kit, Valencia, Calif. was used to extract RNA from sorted cells. The Smart-Seq V4 ultralow input RNA-Seq kit (Clontech) was used to make libraries, which were then sequenced on an Illumina Hiseq (150 bp paired end reads, 26 million paired end reads per sample).

The Galaxy server (usegalaxy.org/) was used for bioinformatic analysis of RNA-Seq data. Nextera paired-ended adapter sequences were removed from the sequencing reads using Trimmomatic (Galaxy version 0.38.0) (minimum quality of Trimmomatic operation=2). Trimmed reads were then aligned to a pseudoatosomal region masked GRCh38 version of the human genome (GCA_000001405.15_GRCh38_no_alt_analysis_set.fna, hgdownload.cse.ucsc.edu/goldenpath/hg38/bigZips/analysisSet/) using TopHat (Galaxy version 2.1.1. A parameter value of zero was used for the mean inner distance between mate pairs for TopHat since the fragment size for these libraries tends to be short relative to the read length used in this study (150 bases paired end reads). The resulting BAM files were sorted using Samtools (Galaxy version 2.0.3) (Li et al., 2009). HTseq (mode=Intersection (nonempty) and ID attribute=gene_name) (Galaxy version 0.9.1) was then used to compute gene counts. The gencode.v31.annotation.gff3.gz annotation file (gencodegenes.org/human/release_31. html) was used for HTseq analysis. With the exception of the parameters noted above, default parameter values were used for Trimmomatic (Bolger et al., 2014), TopHat (Kim et al., 2013) and HT-Seq (Anders et al., 2015) analyses.

DESeq2 was used to perform BCL11B vs. control differential expression analysis (false discovery rate, FDR<0.05). CB Donor identity (CB1-5) and culture time-point (48 hours or 7 days) were co-variates for the multivariate BCL11B vs. Control analysis. CB donor identity (CB1 or CBS) was a co-variate for the BCL11B vs. Control analysis of day 7 samples.

Genes upregulated in BCL11B or control cells in the multivariate or day 7 differential expression analysis, respectively, served as genesets for geneset enrichment (GSEA v4.0) (Subramanian et al., 2005). Enrichment of these genesets was tested among datasets consisting of genes ranked based on fold change observed in Thy1 vs. Thy3 or BCL11B knockdown vs. control shRNA transduced cells DEseq2 differential expression analysis (Love et al., 2014). Published Thy1, Thy3, BCL11B knockdown, and control shRNA RNA-Seq data (Casero et al., 2015, Ha et al 2017) for were used for these differential expression analyses.

Quantitative PCR. The Arcturus Picopure RNeasy micro and the superscript vilo cDNA Synthesis kits (Thermofisher) were used to extract RNA and synthesize cDNA respectively as per manufacturer's instructions. Quantitative PCR (qPCR) was performed using the following TaqMan assays: Hs00256257_m1, (BCL11B), Hs01556515_m1 (TCF7), Hs01062241_ml (CD3E), Hs01547250_m1 (LEF1), Hs00178427_m1 (LCK), and Hs01060665_g 1, (ACTB).

Statistical analysis. We generated second order polynomial repeated measures regression models of the logit of the proportion of cells at a given stage of differentiation vs. time in weeks. Two models were generated for each differentiation stage, namely a model that included the cell type variable (BCL11B vs. control) as a predictor and one that did not include the cell type variable as a predictor. Models were generated for each of the following differentiation stages: CD4+CD3−CD8−, CD4+CD8+CD3−, CD3+CD8+CD4−, CD7+CD1a+, and CD7+CD1a−. The two models for a given stage of differentiation were compared via ANOVA to determine whether the kinetics of differentiation were different between BCL11B and control cells. Second order polynomial repeated measures regression of the logit of the proportion of CD4+CD8+CD3− cells observed in ATO vs. time in weeks was performed separately on data from BCL11B or control cells respectively to generate the differentiation kinetics curves shown in FIG. 3C.

A two-sided paired t-test on log₁₀ transformed cell counts was used to compare cell counts of CD7+CD1a+, CD4+CD8+, or CD8SP cells generated in ATO initiated with BCL11B or control cells. A linear mixed effects model that included variation from the cord blood donor as a random effect, and time and cell type (BCL11B vs. control) variables as fixed effects, was used to compare cell output in the T-cell activation assay between BCL11B and control cells. A two-sided paired t-test on logit transformed proportions was used to compare frequencies of CD33+ cells in MS5 organoids between organoids initiated with BCL11B and control cells, and to compare frequencies of CD45RO+CCR7+CD62L+ cells between cultures initiated with naïve T-cells derived from BCL11B and control ATOs.

It will be apparent that the precise details of the methods described herein may be varied or modified without departing from the spirit of the described embodiments. We claim all such modifications and variations that fall within the scope and spirit of the claims below.

We claim:

1. A method of treating a subject with a T cell therapy, comprising:
providing hematopoietic stem and progenitor cells (HSPCs), pluripotent stem cells, or mature T cells;
increasing BCL11B expression in the HSPCs, pluripotent stem cells, or mature T cells to form modified cells with increased BCL11B expression compared to corresponding control cells, wherein the increased BCL11B expression increases production and/or proliferation of T cells from the HSPCs or the pluripotent stem cells, or increases proliferation of the mature T cells, compared to the corresponding control cells; and
administering a therapeutically effective amount of the modified cells to the subject for the T cell therapy.

2. The method of claim 1, wherein the subject is a hematopoietic stem cell transplant (HSCT) patient and the T cell therapy comprises thymic T cell reconstitution in the subject following the HSCT.

3. The method of claim 2, wherein the modified cells are administered to the subject with the HSCT.

4. The method of claim 1, wherein the T cell therapy is a chimeric antigen receptor (CAR) T cell therapy, and the method further comprises transducing the HSPCs, pluripotent stem cells, mature T cells, or the modified cells with a heterologous nucleic acid molecule encoding the CAR before administering the cells to the subject.

5. The method of claim 1, wherein the T cell therapy is an engineered T cell receptor (TCR) T cell therapy, and the method further comprises transducing the HSPCs, pluripotent stem cells, mature T cells, or the modified cells with a heterologous nucleic acid molecule encoding the TCR before administering the cells to the subject.

6. The method of any one of the prior claims, further comprising incubating the modified cells in vitro under conditions sufficient for differentiation and proliferation of T cells from the HSPCs and/or pluripotent stem cells, or proliferation of the mature T cells, prior to administering the cells to the subject.

7. A method of producing a T cell population for a T cell therapy for a human subject, comprising:
providing hematopoietic stem and progenitor cells (HSPCs), pluripotent stem cells, or mature T cells;
increasing BCL11B expression in the HSPCs, pluripotent stem cells, or mature T cells to form modified cells with increased BCL11B expression compared to corresponding control cells; and
wherein the increased BCL11B expression increases production and/or proliferation of T cells from the HSPCs or the pluripotent stem cells, or increases proliferation of the mature T cells, compared to the corresponding control cells, to form the T cell population for the T cell therapy.

8. The method of claim 7, further comprising incubating the modified cells in vitro under conditions sufficient for differentiation and proliferation of T cells from the HSPCs and/or pluripotent stem cells, or proliferation of the mature T cells, to form the T cell population for the T cell therapy.

9. The method of claim 8, wherein the modified cells are incubated in vitro for more than 14 days under conditions sufficient for the differentiation and proliferation of T cells from the HSPCs or the pluripotent stem cells, or the proliferation of the mature T cells.

10. The method of claim 8, wherein the modified cells are incubated in vitro for more than 30 days under conditions sufficient for the differentiation and proliferation of T cells from the HSPCs or the pluripotent stem cells, or the proliferation of the mature T cells.

11. The method of any one of claims 7-10, wherein the subject is a hematopoietic stem cell transplant (HSCT) patient and the T cell therapy comprises thymic T cell reconstitution in the subject following the HSCT.

12. The method of any one of claims 7-10, wherein the T cell therapy is a chimeric antigen receptor (CAR) T cell therapy, and the method further comprises transducing the HSPCs, pluripotent stem cells, mature T cells, or the modified cells with a heterologous nucleic acid molecule encoding the CAR.

13. The method of any one of claims 7-10, wherein the T cell therapy is an engineered T cell receptor (TCR) T cell therapy, and the method further comprises transducing the HSPCs, pluripotent stem cells, mature T cells, or the modified cells with a heterologous nucleic acid molecule encoding the TCR.

14. The method of any one of the prior claims, further comprising obtaining the HSPCs, pluripotent stem cells, or mature T cells from the human subject.

15. The method of any one of the prior claims, wherein BCL11B expression level in the modified cells is at least that of a control CD34+ or CD34−CD4+CD8+ human thymic T-cell precursor.

16. The method of any one of the prior claims, comprising increasing BCL11B expression level 2 to 10-fold in the mature T cells compared to the BCL11B expression level in corresponding control cells.

17. The method of any one of the prior claims, wherein increasing BCL11B expression comprises transducing the HSPCs, pluripotent stem cells, or mature T cells with a heterologous nucleic acid encoding BCL11B.

18. The method of claim 17, comprising transducing the HSPCs, pluripotent stem cells, or mature T cells with a viral vector comprising the nucleic acid encoding for BCL11B operably linked to a promoter.

19. The method of claim 18, wherein the viral vector is a lentiviral vector.

20. The method of claim 17 or claim 18, wherein the promoter is an MND promoter or a MSCV promoter.

21. The method of any one of claims 17-20, wherein the HSPCs, pluripotent stem cells, or mature T cells are transduced at a multiplicity of infection of between 1 and 10.

22. The method of claim 21, wherein the HSPCs, pluripotent stem cells, or mature T cells are transduced at a multiplicity of infection of between 1 and 5.

23. The method of any one of the prior claims, wherein increasing BCL11B expression in the HSPCs or pluripotent stem cells increases the rate of production of T cells from the HSPCs or the pluripotent stem cells compared to corresponding control cells without the increased BCL11B expression.

24. The method of any one of the prior claims, wherein T cells proliferating from the modified cells have delayed exhaustion in the subject compared to corresponding control cells without the increased BCL11B expression.

25. The method of any one of the prior claims, wherein the subject is a human and the HSPCs, pluripotent stem cells, or mature T cells are human cells.

26. The method of any one of the prior claims, wherein T cells proliferating from the modified cells have an increased central memory immunophenotype compared to control cells without the increased BCL11B expression.

27. The method of claim 26, wherein the T cells with the increased central memory immunophenotype are CD45RO+CD62L+CCR7+ T cells.

28. The method of any one of the prior claims, wherein T cells proliferating from the modified cells have increased interleukin 2 production and/or TNF-alpha production compared to control cells without the increased BCL11B expression.

29. The method of any one of the prior claims, wherein T cell proliferation from the modified cells is independent of Notch signaling.
